SEQUENCE LISTING (17 sequences)

SEQ ID NO: 1 - DNA, 25 bp, Artificial Sequence
  Description of Artificial Sequence Synthetic primer
gattcaataggttgtatgcatggtt

SEQ ID NO: 2 - DNA, 25 bp, Artificial Sequence
  Description of Artificial Sequence Synthetic primer
ttcaggcacacaaacttgatgggcg

SEQ ID NO: 3 - DNA, 181 bp, Artificial Sequence
  Description of Artificial Sequence Synthetic polynucleotide
gattcaataggttgtatgcatggttcatccgaaccggatttgagaaactggaaatcgccaaccccccagttcactcaaggagcccggccggatgaacacccataagcatgcccgattgaccttcctacgtcgactcgaaatggtccagcaattgatcgcccatcaagtttgtgtgcctgaa

SEQ ID NO: 4 - DNA, 80 bp, Artificial Sequence
  Description of Artificial Sequence Synthetic oligonucleotide
ctacctacgatctgactagcnnnnnnnnnnnnnnnnnnnnnnnnnnnnnnnnnnnnnnnngcttactctcatgtagttcc

SEQ ID NO: 5 - DNA, 20 bp, Artificial Sequence
  Description of Artificial Sequence Synthetic primer
ctacctacgatctgactagc

SEQ ID NO: 6 - DNA, 20 bp, Artificial Sequence
  Description of Artificial Sequence Synthetic primer
ggaactacatgagagtaagc

SEQ ID NO: 7 - DNA, 97 bp, Artificial Sequence
  Description of Artificial Sequence Synthetic oligonucleotide
gcctgttgtgagcctcctgtcgaannnnnnnnnnnnnnnnnnnnnnnnnnnnnnnnnnnnnnnnnnnnnnnnnnttgagcgtttattcttgtctccc

SEQ ID NO: 8 - DNA, 78 bp, Artificial Sequence
  Description of Artificial Sequence Synthetic oligonucleotide
gcctgttgtgagcctcctgtcgaagcacgtttatccgtccctcctagtggcgtgcttgagcgtttattcttgtctccc

SEQ ID NO: 9 - DNA, 24 bp, Artificial Sequence
  Description of Artificial Sequence Synthetic primer
gcctgttgtgagcctcctgtcgaa

SEQ ID NO: 10 - DNA, 23 bp, Artificial Sequence
  Description of Artificial Sequence Synthetic primer
gggagacaagaataaacgctcaa

SEQ ID NO: 11 - DNA, 87 bp, Artificial Sequence
  Description of Artificial Sequence Synthetic oligonucleotide
gcctgttgtgagcctcctgtcgaannnnnnnnnnnnnnnnnnnnnnnnnnnnnnnnnnnnnnnnttgagcgtttattcttgtctccc

SEQ ID NO: 12 - DNA, 158 bp, Artificial Sequence
  Description of Artificial Sequence Synthetic polynucleotide
cctcaccaccaacttcatccacgttcaccttgccccacagggcagtaacggcagacttctccacaggagtcagatgcaccatggtgtctgtttgaggttgctagtgaacacagttgtgtcagaagcaaatgtaagcaatagatggctctgccctgact

SEQ ID NO: 13 - DNA, 158 bp, Artificial Sequence
  Description of Artificial Sequence Synthetic polynucleotide
cctcaccaccaacttcatccacgttcaccttgccccacagggcagtaacggcagacttctcctcaggagtcagatgcaccatggtgtctgtttgaggttgctagtgaacacagttgtgtcagaagcaaatgtaagcaatagatggctctgccctgact

SEQ ID NO: 14 - DNA, 20 bp, Artificial Sequence
  Description of Artificial Sequence Synthetic primer
agtcagggcagagccatcta

SEQ ID NO: 15 - DNA, 20 bp, Artificial Sequence
  Description of Artificial Sequence Synthetic primer
cctcaccaccaacttcatcc

SEQ ID NO: 16 - DNA, 15 bp, Artificial Sequence
  Description of Artificial Sequence Synthetic primer
acggcagacttctcc

SEQ ID NO: 17 - DNA, 17 bp, Artificial Sequence
  Description of Artificial Sequence Synthetic primer
gataggactcatcacca

(19) United States
(12) Patent Application Publication
(10) Pub. No.: US 2014/0295424 A1
(43) Pub. Date: Oct. 2, 2014

(54) Isolation and Enrichment of Nucleic Acids on Microchip

(71) Applicant: The Tustees of Columbia University in the City of New York, New York, NY (US)

(72) Inventors: Qiao Lin, New York, NY (US); Jing Zhu, New York, NY (US); Jinho Kim, New York, NY (US); John Paul Hilton, New York, NY (US); Renjun Pei, Suzhou (CN); Milan N. Stojanovic, Fort Lee, NJ (US)

(73) Assignee: The Tustees of Columbia University in the City of New York, New York, NY (US)

(21) Appl. No.: 14/221,596

(22) Filed: Mar. 21, 2014

Related U.S. Application Data
(63) Continuation of application No. PCT/US12/56888, filed on Sep. 24, 2012.
(60) Provisional application No. 61/538,774, filed on Sep. 23, 2011.
(60) Provisional application No. 61/542,124, filed on Sep. 30, 2011.
(60) Provisional application No. 61/588,078, filed on Jan. 18, 2012.
(60) Provisional application No. 61/588,079, filed on Jan. 18, 2012.
(60) Provisional application No. 61/588,082, filed on Jan. 18, 2012.
(60) Provisional application No. 61/590,458, filed on Jan. 25, 2012.
(60) Provisional application No. 61/674,187, filed on Jul. 20, 2012.
(60) Provisional application No. 61/674,191, filed on Jul. 20, 2012.
(60) Provisional application No. 61/674,192, filed on Jul. 20, 2012.
(60) Provisional application No. 61/683,977, filed on Aug. 16, 2012.

(51) Int. Cl. C12Q 1/68

(52) U.S. Cl. CPC C12Q 1/6837; C12Q 1/686
USPC 435/6.11; 435/303.1

(57) ABSTRACT

Techniques for isolating, enriching, and/or amplifying target DNA molecules using MEMS-based microdevices are disclosed. The techniques can be used for detecting single nucleotide polymorphism, and for isolating and enriching desired DNA molecules, such as aptamers.

### CROSS REFERENCES TO RELATED APPLICATIONS

This application is a continuation application from PCT/US12/056,888, filed Sep. 24, 2012, which claims priority from U.S. Provisional Application No. 61/538,774, filed Sep. 23, 2011; Provisional Application No. 61/542,124, filed Sep. 30, 2011; Provisional Application No. 61/588,078, filed Jan. 18, 2012; Provisional Application No. 61/588,079, filed Jan. 18, 2012; Provisional Application No. 61/588,082, filed Jan. 18, 2012; Provisional Application No. 61/590,458, filed Jan. 25, 2012; Provisional Application No. 61/674,187, filed Jul. 20, 2012; Provisional Application No. 61/674,191, filed Jul. 20, 2012; Provisional Application No. 61/674,192, filed Jul. 20, 2012; and Provisional Application No. 61/683,977, filed Aug. 16, 2012, the disclosure of each of which is incorporated herein in its entirety.

### STATEMENT REGARDING FEDERALLY FUNDED RESEARCH

This invention was made with government support under CBET-0854030, awarded by the National Science Foundation; RR025816-02 and CA147925-01, both awarded by the National Institutes of Health. The government has certain rights in this invention.

### BACKGROUND

Chemical amplification of nucleic acids can be realized with the polymerase chain reaction (PCR), in which a DNA molecule (a template) can be duplicated via repeated thermal denaturation and enzymatic replication. Bead-based PCR is a variant of the PCR procedure that uses primers (short DNA fragments complementary to a specific region of the template) attached to microbeads. This procedure can result in bead-tethered template DNA duplicates. Therefore, it can serve as an analytical tool to simultaneously accumulate signals from DNA-based transducers and allow manipulation of DNA itself via solid-phase extraction (SPE) techniques.

Bead-based PCR has been used in applications including DNA sequencing, protein screening, and pathogenic DNA detection. For example, whole genome sequencing has been performed using bead-based PCR to facilitate the organization and detection of amplified sections of a fragmented E. coli genome. Compartmentalization of DNA in emulsions combined with bead-based PCR can allow for rapid screening of an entire genome for DNA binding proteins and cell-free protein synthesis.

Microfluidics technology can provide a rapid and efficient reaction platform due to efficient heat transfer properties. Microfluidics can also enable integrated chip-based systems that perform tasks such as sample pretreatment and post-amplification analysis, thereby improving reaction speed and test accuracy by shifting more operations to the microscale domain.

In bioanalytical assays, analytes of interest can be present in minute quantities and contaminated with impurities. Thus, sample preparation steps prior to analysis can be important for improving the resolution of detection results. In particular, isolation and enrichment of DNA molecules within dilute and complex samples can enable clinical detection of DNA markers linked to disease and synthetic selection of analyte-specific molecules such as aptamers.

Aptamers are oligonucleotides that display affinity for target molecules such as proteins, small molecules, nucleic acids, and whole cells, and can have applications to clinical diagnostics and therapeutics. The recognition abilities of aptamers have been employed with various transduction methods to generate novel diagnostic tools. In addition, aptamers have contributed to advances in therapeutics for diseases such as macular degeneration and various types of cancer. “Smart” aptamers can be generated which bind with specific equilibrium constants, kinetic parameters, and at specific temperatures.

Aptamer sequences can be developed by an evolutionary process known as Systematic Evolution of Ligands by Exponential Enrichment, or SELEX. However, it can be labor-intensive, and inefficient. Microchip-based devices for sample enrichment can reduce sample consumption and shorten assay times. Consequently, enrichment techniques can be implemented in microfluidic devices to separate and enrich low-Active concentration biological molecules from complex samples, for example, to improve various aspects of the SELEX process.

Genetic mutations take many forms, ranging from chromosome anomalies to single-base substitutions. Among them, single nucleotide polymorphisms (SNPs), which are single nucleotide variations in the genome between different individuals, are the most common form, occurring approximately once every 1000 bases. SNPs can be used as genetic markers to identify genes associated with complex disease. Therefore, accurate identification of SNPs can be of utility to disease diagnosis and prognosis.

Genotyping of SNPs can be based on enzymatic cleavage, allele specific hybridization, allele specific ligation or cleavage, and allele specific primer extension. Enzymatic cleavage can utilize thermostable flap endonucleases (FEN) and fluorescence resonance energy transfer (FRET) to recognize and detect SNP by the annealing of allele-specific overlapping oligonucleotides to the target DNA. This method is generally time-consuming and difficult to multiplex (i.e., to detect multiple SNPs in one reaction). There is therefore a need for new genotyping platforms to address these issues and offer improved accuracy, ability to multiplex, and increased throughput.

### SUMMARY

The disclosed subject matter provides techniques for isolation, selection, and amplification of nucleic acids, e.g., DNA molecules.

In certain embodiments, a method for amplifying a target DNA molecule using at least a first microchamber is provided. The microchamber can be formed as part of a MEMS-based microdevice, and can include at least one first primer immobilized on a solid phase. The first primer is suitable for amplifying the target DNA. A sample including the target DNA molecule can be introduced into the first microchamber, where the target DNA is hybridized onto the first primer. A complementary DNA of the target DNA can be produced in the first microchamber using the target DNA as a template, e.g., by using a PCR process and suitable PCR reagents and polymerase. The target DNA can then be separated from the complementary DNA. A second primer can be hybridized onto the complementary DNA, e.g., at a free end of the complementary DNA.

An amplification of the target DNA can be obtained using the complementary DNA as a template. Such amplified copy of the target DNA can be again separated from the complementary DNA, and the thermal cycling procedure repeated to produce a plurality of double-stranded DNA each including a copy of the target DNA and a copy of the complementary DNA.

In certain embodiments, the second primer can include a spectroscopically detectable tag, such as a fluorophore. Such a tag can be detected using, e.g., fluorescent spectroscopy. In some embodiments, the target DNA can be an aptamer.

In some embodiments, before introducing the sample containing the target DNA into the chamber for amplification, the sample can be purified. For example, a sample containing the target DNA and non-target DNA molecules can be into a second microchamber which includes an immobilized functional molecule that binds with the target DNA, such that the target DNA binds with the immobilized functional molecule in the second microchamber. The DNA molecules not bound with the functional molecule can be removed, e.g., by washing, and then the bound target DNA can be isolated from the functional molecule. The isolation can be performed by changing the temperature of the second chamber, e.g., raising the temperature. Alternatively, the isolation can be achieved using a chemical reagent, such as an alkali solution.

In certain embodiments, the target DNA can be transported from one microchamber to another microchamber electrophoretically, e.g., via a microchannel that connects the two microchambers and includes a gel suitable for electrophoresis of the target DNA. The transported target DNA can be amplified, using a PCR process, in the latter microchamber on-chip, and the amplified target DNA can be transported back into the former microchamber, e.g., electrophoretically via the same microchannel or another channel including a gel suitable for electrophoresis of the target DNA.

In some embodiments, the target DNA includes at least one polymorphic site, and the method further includes detecting such polymorphic site. After the amplification of the target DNA in a microchamber according to a bead-based PCR procedure (e.g., through multiple thermal cycles), the amplified copy of the target DNA can be separated from the complementary DNA. At least one an allele specific primer can be introduced into the microchamber, such that the primer anneals adjacent to a site of the complementary DNA corresponding to the polymorphic site. The allele specific primer can then be extended by one base to obtain an extended primer. The extended primer can then be isolated from the complementary DNA. The one base included in the isolated extended primer can then be detected, such as by MALDI-TOF mass spectroscopy, thereby determining the identity of the polymorphic site of the target DNA. The target DNA can include a plurality of polymorphic sites. In such a case, a plurality of allele specific primers can be used, each to anneal adjacent to one of the plurality of polymorphic sites. The multiple primers each can have different molecular weight. In such a manner, multiple polymorphic sites can be detected simultaneously.

In other embodiments, detecting a polymorphic site in a target DNA can be achieved as follows, using a microfluidic device having a first microchamber and a second microchamber in fluidic communication with the first microchamber. A sample including the target DNA is introduced into the first microchamber. At least one allele specific primer is introduced to anneal immediately adjacent to the polymorphic site of the target DNA. The allele specific primer is then extended by one base to obtain an extended primer. A plurality of copies of the extended primer are then generated in the first microchamber by one or more thermal cycles. The plurality of copies of the extended primer are transferred into the second microchamber which includes a solid phase having surface-attached functional molecules that bind with the extended primer such that the at least one of the plurality of copies of extended primer is captured by the solid phase.

The captured extended primer can then be isolated from the solid phase, e.g., by chemical cleavage; and the one base included in the isolated extended primer can be detected, e.g., using MALDI-TOF mass spectroscopy, to determine the identity of the polymorphic site of the target DNA. The target DNA can include a plurality of polymorphic sites. In such a case, a plurality of allele specific primers can be used, each to anneal adjacent to one of the plurality of polymorphic sites. The multiple primers each can have different molecular weight. In such a manner, multiple polymorphic sites can be detected simultaneously according to the above procedure.

The disclosed subject matter also provide microdevices, and fabrication methods thereof, for implementing the techniques described above.

### BRIEF DESCRIPTION OF THE DRAWINGS

FIG. 1a is a flowchart of an example process for isolating and amplifying a target DNA according to some embodiments of the disclosed subject matter.

FIG. 1b is a schematic diagram of a system for isolating and amplifying a target DNA according to some embodiments of the disclosed subject matter.

FIGS. 2a-2e is a schematic diagram illustrating a process for isolating and enriching a target DNA from a library of DNAs using a microdevice having an isolation microchamber and an amplification microchamber according to some embodiments of the disclosed subject matter.

FIG. 3 is a schematic diagram illustrating a process for isolating and enriching a target DNA from a library of DNAs using a microdevice having an isolation microchamber, an enrichment microchamber, and a channel connecting the two chambers which include a gel according to some embodiments of the disclosed subject matter.

FIGS. 4a-4d are schematic diagrams illustrating a process of detecting a polymorphic site on a DNA using a single chamber of a microdevice according to some embodiments of the disclosed subject matter.

FIG. 5 is a schematic diagram illustrating an alternative process of detecting a polymorphic site on a DNA using a microdevice having multiple chambers according to some embodiments of the disclosed subject matter.

FIGS. 6a and 6b are schematic diagrams of the structure and dimensions of an example microdevice according to some embodiments of the disclosed subject matter.

FIG. 7 is a plot of a temperature history in a test using the microdevice as depicted in FIG. 6a according to one embodiment of the disclosed subject matter.

FIG. 8 is a plot showing the effect of temperature on fluorescence measurement (n=3, n is the number of tests) in one embodiment of the disclosed subject matter.

FIGS. 9a and 9b are images of gel electrophoresis analysis of tests using an 181 bp DNA segment of the B. pertussis genome, where (a) shows the results of a solution based test and (b) shows the results of a bead-based test according to an embodiment of the disclosed subject matter.

FIG. 10 is a plot showing the effect of annealing temperature on fluorescent intensity after bead-based PCR (n=3) in one embodiment of the disclosed subject matter.

FIG. 11 is a gel electropherogram showing effects of annealing temperature on amplified DNA following conventional solution-based PCR as a comparison with the bead-based PCR.

FIG. 12 is a plot showing the effect of bead concentration on fluorescent bead intensity following PCR in one embodiment of the disclosed subject matter.

FIG. 13 is a plot of fluorescent intensity of beads following the PCR against concentration of templates in the reaction mixture according to one embodiment of the disclosed subject matter. Error bars indicate one standard deviation from the mean of three examples (n=3), and the reaction with zero templates (control) and a 1 pM template concentration (the detection limit) is differentiable with a probability of >95%, according to the Student's t test.

FIG. 14 is a plot showing the relationship between signal intensity and the number of PCR cycles according to one embodiment of the disclosed subject matter. Mean values from multiple tests (n=3) are shown, error bars indicate standard deviation.

FIGS. 15a-15c are micrographs of the microchamber illustrating the process of bead-based PCR according to one embodiment of the disclosed subject matter.

FIG. 16 is a schematic diagram of a microdevice including a selection chamber and an amplification chamber according to some embodiments of the disclosed subject matter.

FIGS. 17a and 17b are images of a microdevice without connections (a), and of the mixer being tested with a dye (b).

FIG. 18 is a gel electropherogram showing off-chip amplification of eluents containing non-target DNA collected in different washes according to some embodiments of the disclosed subject matter.

FIG. 19 is a gel electropherogram showing on-chip amplification of eluents containing target DNA collected in different washes according to some embodiments of the disclosed subject matter.

FIGS. 20a and 20b are plots showing binding affinity comparison between enriched DNA and the starting random library according to some embodiments of the disclosed subject matter.

FIG. 21 is a plot showing the temperature-dependent binding of enriched DNA based on fluorescence measurements according to some embodiments of the disclosed subject matter.

FIG. 22 is a schematic diagram of a microdevice for target DNA isolation and enrichment measurements according to some embodiments of the disclosed subject matter.

FIGS. 23a-23j are schematic diagrams of an example fabrication process for the microchip as shown in FIG. 22.

FIG. 24 is a photograph of a fabricated microdevice illustrated in FIG. 22.

FIG. 25 is an example test setup for operating a microdevice shown in FIG. 24.

FIG. 26a is gel electropherogram of amplified eluents containing non-target DNA as obtained during the isolation process according to one embodiment of the disclosed subject matter; FIG. 26b is a bar graph depicting band intensity for different lanes according to the embodiment of the disclosed subject matter.

FIG. 27a is gel electropherogram of amplified eluents obtained during in a control example; FIG. 27b is a bar graph depicting band intensity for different lanes for the control example.

FIG. 28 depicts electrophoresis of target DNA through the gel-filled microchannel using different electrolytes according to some embodiments of the disclosed subject matter.

FIGS. 29a-29d depict electrophoretic transport of fluorescently labeled target DNA through the gel-filled microchannel under an electric field of 25 V/cm at different times according to some embodiments of the disclosed subject matter.

FIG. 30 is a gel electropherogram of eluents obtained from the isolation and enrichment chambers after one round of isolation and enrichment according to some embodiments of the disclosed subject matter.

FIG. 31a is a gel electropherogram of eluents obtained from the enrichment chamber following PCR amplification; FIG. 31b is a bar graph depicting band intensities of the eluents according to some embodiments of the disclosed subject matter.

FIG. 32 is a schematic diagram of an example microdevice according to one embodiment of the disclosed subject matter.

FIG. 33 is a photograph of an example microdevice (with the chambers and channels filled with colored ink for visualization) according to one embodiment of the disclosed subject matter.

FIG. 34a is a gel electropherogram of eluents obtained during the isolation according to one embodiment of the disclosed subject matter; FIG. 34b is a bar graph showing band intensity profile for incubation, washes, and elution samples according to the embodiment of the disclosed subject matter.

FIG. 35a is a gel electropherogram of eluents obtained during the isolation according to a control example; FIG. 35b is a bar graph showing hand intensity profile for incubation, washes, and elution samples according to the control example.

FIG. 36a-36c show electrophoretic transport of fluorescently labeled target DNA under an electric field of 25 V/cm at different times according to one embodiment of the disclosed subject matter; FIG. 36d shows fluorescence intensity as a function of time monitored at the detection site according the embodiment of the disclosed subject matter.

FIG. 37 is a gel electropherogram of eluents obtained from the isolation and enrichment chambers after one round of isolation and enrichment example according one embodiment of the disclosed subject matter.

FIG. 38a is a gel electropherogram of eluents obtained from the enrichment chamber following PCR amplification according one embodiment of the disclosed subject matter; FIG. 38b is a bar graph showing band intensities of the eluents for increasing rounds of enrichments according the embodiment of the disclosed subject matter.

FIG. 39 is an image of an example microdevice (filled with colored ink for visualization) according to one embodiment of the disclosed subject matter.

FIG. 40a is a gel electropherogram of amplified eluents containing non-target DNA as obtained from the selection chamber in an example using the microdevice depicted in FIG. 39; FIG. 40b is a bar graph showing corresponding fluorescent intensity of the lanes in FIG. 40a.

FIG. 41a is a micrograph of beads in the PCR chamber according to one embodiment of the disclosed subject matter; FIGS. 41b and 41e are fluorescence images of the beads before (in (b)) and after (in (c)) hybridization of fluorophore-labeled target DNA (Scale bar in (a)-(c): 100 μm) according to the embodiment; FIG. 41d is a bar graph depicting the fluorescence intensities of the beads according to the embodiment of the disclosed subject matter.

FIGS. 42a-42c are fluorescence images of beads after (a) 0, (b) 10, (c) 20 PCR cycles according to some embodiments of the disclosed subject matter (scale bar: 100 μm.); FIG. 42d is a bar graph showing corresponding fluorescence intensities of the beads.

FIG. 43 is a bar graph showing binding affinities of enriched DNA and random DNA to IgE-coated beads according to some embodiments of the disclosed subject matter.

FIGS. 44a and 44b are schematic diagrams of the structure of a microdevice according to some embodiments of the disclosed subject matter; FIGS. 44c-44g are schematic diagrams of an example process for fabricating the microdevice of FIGS. 44a and 44b. All dimensions shown are in microns.

FIG. 45 is a photograph of a fabricated microdevice as schematically shown in FIGS. 44a and 44b.

FIG. 46 is an example test setup for using the microdevice as depicted in FIG. 45 in detecting a polymorphic site of a DNA.

FIG. 47 is a plot of history of chamber temperature in a calibration test using the microdevice as depicted in FIG. 45.

FIG. 48a is a bar graph showing characterization of bead-based PCR in a genotyping example using the microdevice as depicted in FIG. 45 by fluorescent intensity of beads with different PCR parameters, measured in arbitrary units (a.u.); FIG. 48b is a bar graph showing the effect upon removal of target DNA from the beads by NaOH (error bars represent standard deviations based on four independent measurements of fluorescent microbeads).

FIG. 49a is a bar graph showing fluorescent intensity of beads before desalting, after desalting and after thermal elution in a genotyping example using the microdevice as depicted in FIG. 45; FIG. 49b is a bar graph in the genotyping example showing fluorescent intensity of FAM-labeled microbeads following heating; FIG. 49c is A MALDI-TOF mass spectrum of thermally eluted FAM-modified forward primers. Error bars in (a) and (b) represent standard deviations based on four independent measurements of fluorescent microbeads.

FIG. 50a is a MALDI-TOF mass spectrum of a mutated HBB gene in a genotyping test using the microdevice as depicted in FIG. 45 for; FIG. 50b is a corresponding MALDI-TOF mass spectrum of an unmutated HBB gene (where asterisks “*” denote extended SBE primer).

FIG. 51 presents exemplary molecular structures of cleavable biotinylated ddNTPs according to some embodiments of the disclosed subject matter.

FIG. 52a is a cross-sectional schematic diagram of an SNP detection device according to one embodiment of the disclosed subject matter; FIG. 52b is a photograph of a fabricated SNP detection device according to one embodiment of the disclosed subject matter.

FIG. 53 is a plot depicting a calibration of a temperature sensor of the SNP detection device shown in FIG. 52b.

FIG. 54a is a plot showing a time-resolved tracking curve in a test of the temperature of SBE chamber of the device shown in FIG. 52b; FIG. 54b is a plot of time-resolved tracking curve in a test of the temperature of SPC channel of the device shown in FIG. 52b.

FIG. 55a is a MALDI-TOF mass spectrum of single base extension product in a test using the SNP detection device depicted in FIG. 52b (inset: structure of extended primer terminated with ddUTP-N3-biotin, the peak marked by ‘*’ is caused by impurities in the commercial synthetic primer); FIG. 55b is a MALDI-TOF mass spectrum of solid phase capture and chemical cleavage product in a test using the SNP detection device depicted in FIG. 52b (inset: structure of the cleaved product); FIG. 55c is a MALDI-TOF mass spectrum of the desalted product in a test using the SNP detection device depicted in FIG. 52b.

FIG. 56 is a MALDI-TOF mass spectrum of SNP detection product with all operations integrated in a test using the SNP detection device depicted in FIG. 52b.

### DETAILED DESCRIPTION

The disclosed subject matter provides techniques for isolation, selection, and amplification of nucleic acids, e.g., DNA molecules on a microchip. More specifically, the disclosed subject matter provides for MEMS-based microdevice platform and associated methods for isolating and enriching desired DNA for genotyping and other applications.

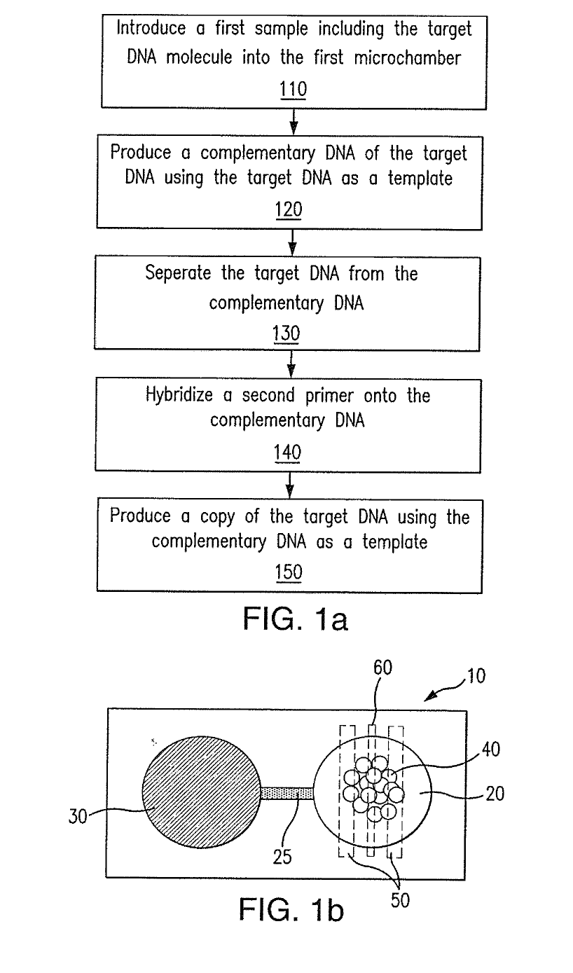

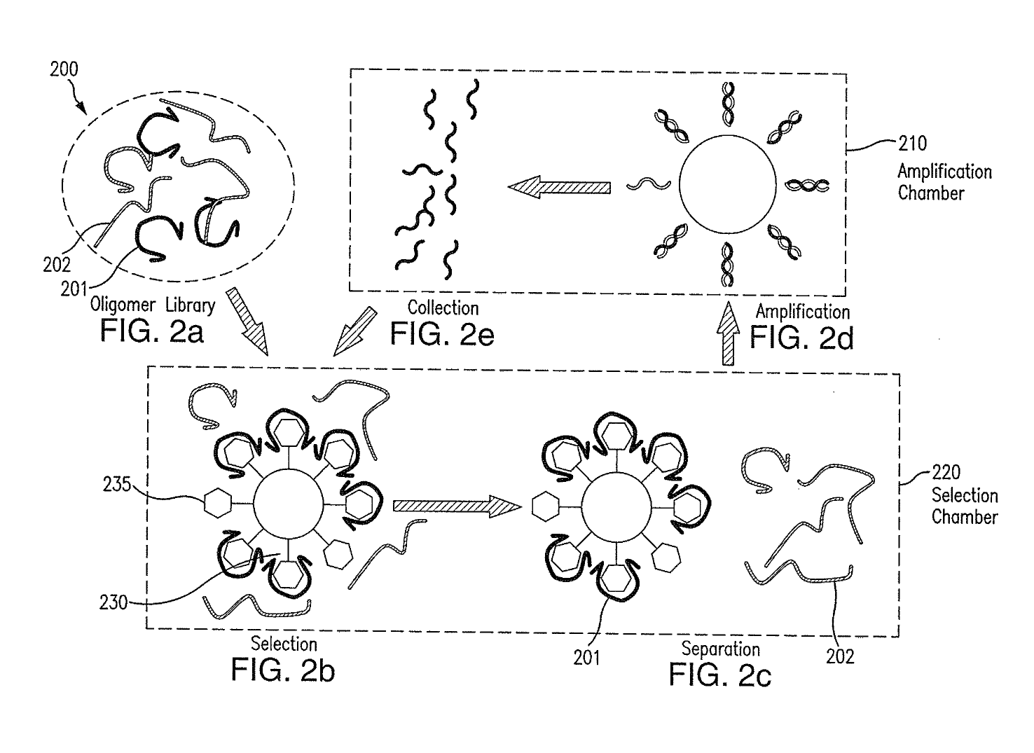

In one aspect, the presently disclosed subject matter provides a method for amplifying a target DNA molecule using a microchamber including a first primer immobilized on a solid phase (e.g., microbeads) in the first microchamber. Referring to FIG. 1a, the method includes introducing a first sample including the target DNA molecule into the first microchamber (at 110), where the target DNA is hybridized onto the first primer which is suitable for amplifying the target DNA. A complementary DNA of the target DNA is produced in the first microchamber using the target DNA as a template (at 120). The target DNA is then separated from the complementary DNA (at 130). A second primer is hybridized onto the complementary DNA (at 140). The target DNA is then amplified using the complementary DNA as a template (at 150).

The above procedure can be performed in a microchamber (or simply “chamber”) of a microdevice (also referred to as microchip) loaded with microbeads as the solid phase. The microdevice can be fabricated using standard microfabrication techniques, e.g., using PDMS soft lithography to create a chamber with desired shape and dimension. For example and not limitation, the microchamber can have a diameter of from about 0.1 mm to about 2 mm, and a depth of about 0.05 to 0.5 mm. Microheaters and temperature sensors, which can be used for temperature regulation in the PCR process, can be integrated into the microdevice, e.g., situated in a thin film layer underneath the microchamber. In connection with this embodiment and other embodiments as further described below, and for illustration and not limitation, FIG. 1b schematically depicts an example microdevice 10 having a microchamber 20 loaded with solid phase 40, the microchamber positioned above microheater 50 and temperature sensor 60. In some embodiments, the microdevice can further include a second microchamber 30 connected with the microchamber 20 via a microchannel 25. Further description of the various features and embodiments of the microdevice and fabrication thereof is provided in the Examples.

The target DNA can be from different sources, including synthetically generated DNA such as a randomized oligonucleotide library, or genomic DNA extracted from cells. Source locations can include off-chip processes as well as on-chip pre-processing of samples.

The microbeads are functionalized with a suitable primer for amplifying the target DNA (also referred to as “template DNA”). The microbeads can be polymer beads coated with streptavidin, which is known to have extraordinarily high affinity for biotin. The primer (e.g., a reverse primer) can be biotin-functionalized and immobilized onto the beads surface. When the sample including the target DNA is introduced into the chamber, the target DNA can hybridize to the bead-immobilized primers due to molecular recognition (e.g., Watson-Crick type base pairing). Other molecules in the sample, such as non-target DNA molecules, cells, small molecules, etc., are less likely to bind with the primers. Using the bead-immobilized primer and PCR reagents (including e.g., Tag polymerase, deoxynucleotide triphosphates, and buffer), a complementary DNA can be produced based on the target DNA, which together with the target DNA forms a double-stranded DNA (ds-DNA) tethered on the beads. Such ds-DNA can be denatured (or melted) at an elevated temperature, e.g., about 95° C., to separate the target DNA from the complementary DNA. A second primer, e.g., a forward primer, can be annealed onto the complementary DNA (e.g., at the free end of the complementary DNA) at a lowered temperature, e.g., at 50-62° C. Thereafter, using the contemporary DNA as a template, the second primer, and the PCR reagents, another copy of the target DNA can be produced, at a suitable chain extension temperature, e.g., about 72° C. Repeating the above temperature cycles (melting, annealing, and extension) can result in amplification of the target DNA, i.e., generation of exponentially increasing duplicate copies of the target DNA.

The untethered second primer can be labeled with a spectroscopically detectable tag (e.g., a fluorophore). In such a case, the result of the amplification after a number of PCR cycles can be fluorophore-labeled target DNA and unlabeled, bead-tethered complementary strands. Such labeled target DNA can be isolated for detection by fluorescent spectroscopy.

The above-described bead-based PCR procedure in a microchamber of a microdevice provides a convenient framework for detecting and manipulating DNA on-chip. Various processes concerning isolation, enrichment, evaluation of DNA utilizing such PCR procedure are contemplated. Some example embodiments are described below.

The sample including the target DNA (and other impurities) can be first processed such that the target DNA can be selectively captured by certain functional molecules that specifically bind with the target DNA. For example, molecular recognition of sequence-specific binding structures allows purification of such sequences from complex mixtures using a molecular analyte. In such a case, the functional molecule can be protein Immunoglobulin E (IgE), and the target DNA can be aptamer(s) specifically binding with IgE. This can be particularly useful for isolating and amplifying a target DNA in a sample which contains various other DNA sequences, e.g., an oligomer library.

An example procedure employing such pre-selection and follow-on amplification is illustrated in FIGS. 2a-2e. The pre-selection can be accomplished using a second chamber (also referred to as “isolation chamber” or “selection chamber”), e.g., situated on the same microdevice and in fluidic communication with the first chamber. The functional molecules can be attached on microbeads loaded in the second chamber. When the sample including an oligomer library 200 (in FIG. 2a) including target DNA 201 and non-target DNA 202 is introduced into the selection chamber 220, the target DNA 201 binds with the immobilized functional molecules 235 attached to the microbead 230 (FIG. 2b). The non-target DNA 202 that are not bound (or weakly bound) with the functional molecules can be removed, e.g., by washing (FIG. 2c). The bound target DNA can then be released from the functional molecules, and transported into the first chamber 210 (also referred to as “amplification chamber” or “enrichment chamber”), e.g., by elution using a buffer solution. The transported target DNA in the amplification chamber can be subject to amplification as described above (FIG. 2d), and the amplification products (increased amounts of the target DNA) can be isolated (FIG. 2e) for detection, or transported back into the selection chamber for further rounds of isolation-amplification).

The target DNA can be an aptamer. Aptamers can be developed for a broad spectrum of analytes with high affinity, possess well controlled target selectivity, and be synthesized to bind targets with predefined binding characteristics, such as temperature-sensitive binding. As such, external stimuli (such as temperature, pH, or ionic content) can be used to disrupt the aptamer-target binding complex. For example, chamber 220 can be set at a first temperature T₁ for binding of the target aptamer, and after the removal of the unbound DNAs and other impurities, the temperature of the second chamber can be changed, e.g., raised to T₂ which is higher than T₁ such that the conformal structure of the aptamer is disrupted, thereby releasing the aptamer from the functional molecules. The temperature control can be achieved by integrated microheater and temperature sensor associated with the selection chamber. For certain aptamers, release temperature T₂ can be lower than the capture temperature T₁. In such cases, the lower temperature T₂ can be achieved by thermoelectric cooling, e.g., by Peltier elements incorporated in the microdevice. Alternatively, the aptamer bound to the functional molecule can also be released using a reagent, such as an alkali solution.

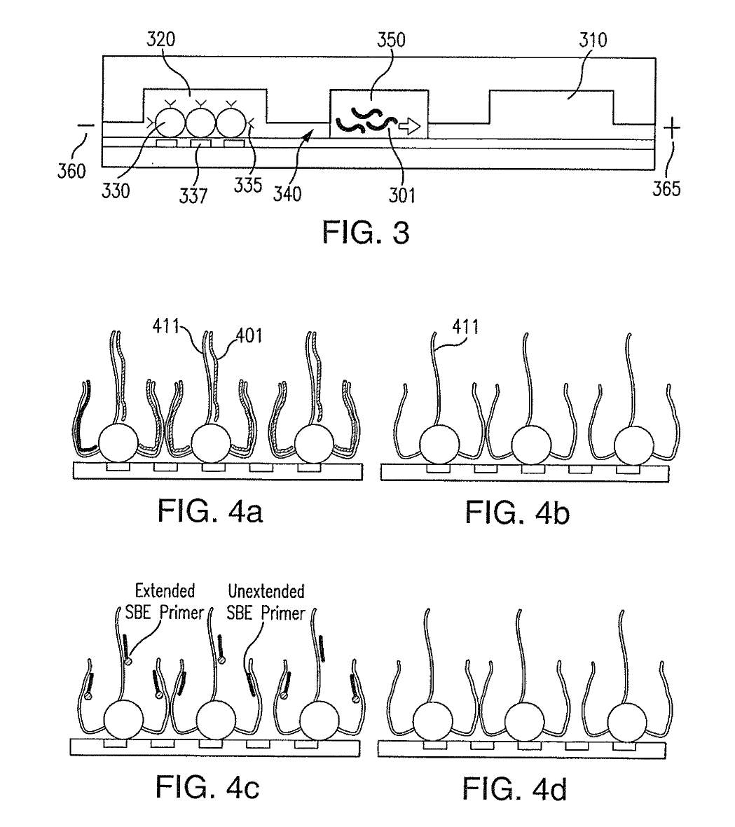

In example embodiments, the transporting of the target DNA from the selection chamber to the enrichment chamber can be accomplished by electrophoresis. As illustrated in FIG. 3, a microchannel 340 connecting the selection chamber 320 and the enrichment chamber 310 includes a section filled with a gel 350. The gel can be any commonly used gel suitable for electrophoresis of DNAs, such as agarose gel. After release (e.g., thermal release, where the heat can be supplied by the microheater 337 beneath the selection chamber 320) of the target DNA 301 from the functional molecule 335 immobilized on beads 330, the target DNA 350 is transported by electrophoresis through the gel 350, the electric field being supplied by a voltage applied between the positive electrode 365 and negative electrode 360. The transportation of the released target DNA only occurs when a suitable electric field is applied through the gel. Thus, such arrangement can provide effective isolation between the enrichment chamber and the selection chamber, and therefore allow independent operation in the selection chamber (e.g., washing, elution) without the risk of contaminating the enriched products in the enrichment chamber.

After the target DNA is transported into the enrichment chamber, further selection-transportation rounds can be performed, such that more target DNA will be accumulated in the enrichment chamber. Additionally or alternatively, the target DNA can be amplified in the enrichment chamber using the above-described procedure. The amplified products can be transported back into the selection chamber for further rounds of selection-transportation-amplification if desired. Such transportation from the enrichment chamber to the selection chamber can be again by electrophoresis, e.g., using the microchannel 340 and the gel 350 loaded therein (and a reverted electric field applied on the electrodes 360 and 365), or via a second gel-filled microchannel connecting the two chambers.

In other embodiments, the on-chip PCR procedure can be employed for detection of a polymorphic site in a target DNA (i.e., DNA having a single nucleotide polymorphism (SNP)), which is illustrated in FIGS. 4a-4d. In such embodiments, a sample including the target DNA 401 having a SNP can be first amplified by bead-based PCR as described above, which results in bead-tethered ds-DNAs including target DNA 401 and complementary DNA 411 (FIG. 4a). The target DNA can be separated from the complementary strands (e.g., by chemical elution or denaturation) and washed off, leaving behind bead-tethered complementary DNA strands 411 (FIG. 4b). Single base extension (SBE) reactants can be then introduced, and allele specific primer can be introduced to anneal immediately adjacent to a site of the complementary DNA corresponding to the site of single nucleotide polymorphism on the template DNA. These primers then undergo SBE, by thermally cycling the reaction mixture in the presence of dideoxynucleotides (ddNTPs) and enzyme, to generate primers extended by only one base (FIG. 4c). The free primers, salts and any other impurities can be washed off for purification of the bead-bound extended and unextended allele-specific primers, followed by thermal or chemical elution of the extended or unextended primers (FIG. 4d). The extended one base included in the isolated extended primer can be detected, e.g., by MALDI-TOF mass spectroscopy, according to the difference in mass between the extended and unextended primers, thereby determining the identity of the polymorphic site of the target DNA.

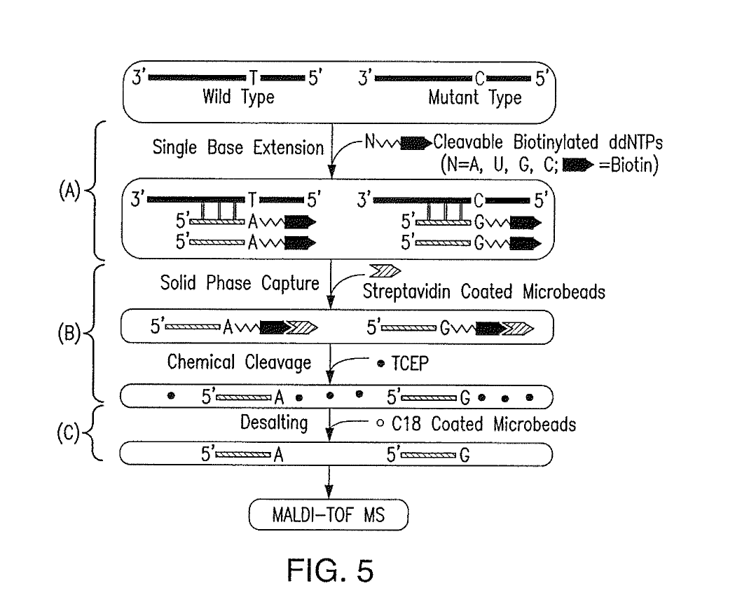

Alternatively, detection of a polymorphic site in a target DNA can be accomplished on a microdevice using different procedures not involving PCR. One example procedure for such detection is illustrated in FIG. 5, where a target DNA including a SNP and a corresponding wild-type DNA are shown side by side for the sequences in the procedure. A sample including the target DNA can be introduced into the first microchamber (“SBE chamber”). SBE reactants (including e.g., cleavable biotinylated ddNTPs) can then be introduced, as well as allele specific primers, which are annealed immediately adjacent to the polymorphic site of the target DNA, whereby the allele specific primers are each extended by one base to obtain extended primers (FIG. 5a). A plurality of copies of the extended primer can be obtained, including free extended primers (not bound to target DNA), after one or more thermal cycles. The free extended primers can be transferred to a microchannel including a solid phase purify the extended primers by perform solid phase capture (SPC). For SPC, the solid phase can have surface-attached functional molecules that specifically bind with the extended primers. For extended primers including biotinylated ddNTP, streptavidin can be used as the functional molecule based on its strong affinity with biotin. The captured extended primers can then be isolated from the solid phase by e.g., chemical cleavage, and the isolated extended primers further desalted and then detected, e.g., by MALDI-TOF mass spectroscopy, according to the difference in mass between the extended and unextended primers, thereby determining the identity of the polymorphic site of the target DNA.

Further details of device structure, fabrication, and operation procedures of the above-described embodiments can be found in the following Examples, which are provided for illustration purpose only and not for limitation.

### Example 1

This example demonstrates bead-based PCR for isolating and amplifying a target DNA on a microchip including integrated heaters and temperature sensors.

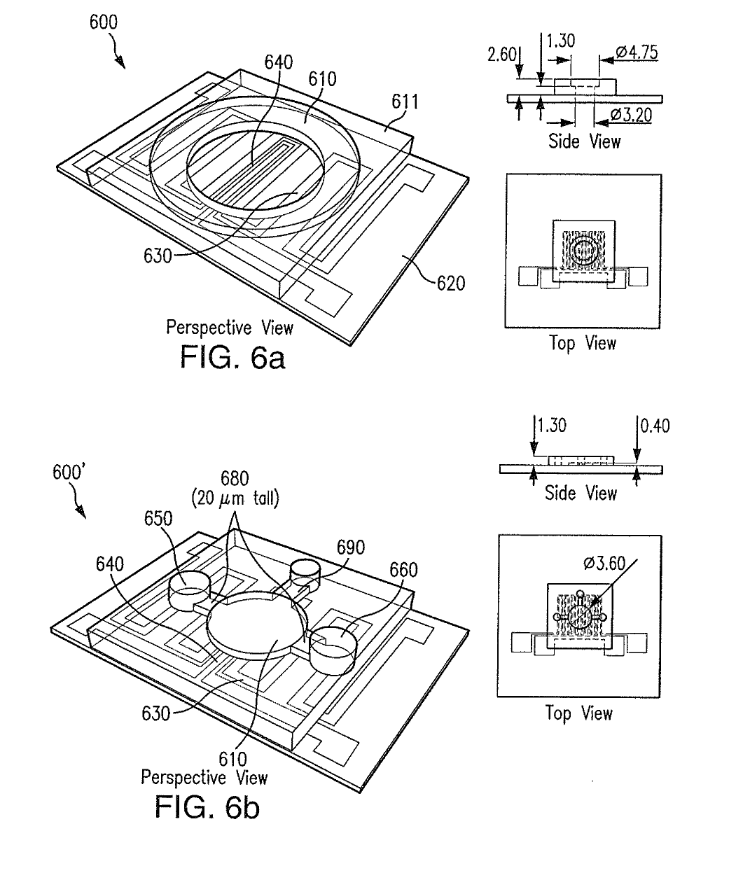

As illustrated in FIG. 6, the bead-based PCR microchip 600 includes a microchamber 610, which was fabricated using polydimethylsiloxane (PDMS) which forms side wall 611 of the microchamber, and bonded to a substrate 620 (a glass slide) with an integrated resistive heater 630 and temperature sensor 640 (see FIG. 6a). The heater 630 has a serpentine geometry covering the chamber area as well as a large surrounding area to generate a sufficiently uniform temperature field in the chamber, while the resistive temperature sensor 640 is located at the center of the chamber area. The cylindrical chamber is open to atmosphere and includes two vertically aligned connected compartments of different diameter. The lower compartment can be used to accommodate reactants (including surface functionalized microbeads, target DNA, PCR reagents, etc.). The upper compartment of larger diameter can be used to retain a layer of mineral oil over the reactants. The inner chamber surface, was coated with a layer of the polymer Parylene C. The mineral oil and Parylene coating can reduce water evaporation that would otherwise occur to open air or through the PDMS, while also reducing the probability of air bubble formation. The Parylene also provides a PCR-compatible surface, which, along with the use of additives such as bovine serum albumin (BSA) and Tween, minimizes adsorption of reaction components such as DNA and Taq polymerase.

Devices were fabricated using standard microfabrication techniques. Chrome and gold layers for heater and temperature sensor (thicknesses 20 and 200 nm) were thermally deposited onto glass microscope slides and patterned using contact lithography and wet etching techniques. Patterning generated a 5.67 cm long by 200 μm wide heater having a resistance of 20Ω and covering an area of 0.242 cm², and a 1.04 cm long by 40 μm wide resistive temperature sensor having a resistance of ˜30Ω. The thermal elements were then passivated with SiO₂ (thickness 1 μm) formed using plasma-enhanced chemical vapor deposition, with openings for electrical connections formed using a shadow mask. The SiO₂ not only passivates the electrical components, but also provides an efficient bonding surface for PDMS. To generate PDMS for the microfluidic chambers, PDMS prepolymer was mixed in the ratio of 10:1 with a curing agent and poured onto a clean silicon wafer, baked for 30 min at 75° C., and then peeled from the wafer.

Microfluidic chambers were defined by puncturing holes in the PDMS using a hole punch. The bottom PDMS piece was 1.3 mm thick with a 3.2 mm diameter hole, and the top piece was 1.3 mm thick with a 4.75 mm diameter hole (FIG. 6a, side view). The glass substrate and PDMS sections were then treated using UV-generated ozone for 10 min, and bonded by baking at 75° C. for 30 min. During bonding, the PDMS holes were aligned to the center of the integrated heater patterned onto the glass slide. The chip was then conformally coated with a layer of Parylene C in thickness via chemical vapor deposition, with scotch tape used to block electrical pads.

In an alternative design, as illustrated in FIG. 6b, the microdevice 600′ also incorporates microfluidic channels enabling bead insertion (via microchannel 680) and retention (via weirs 680) as well as ports for introducing reactants and buffer solutions during wash steps (including ports 650 and 660). To fabricate such a device, optical lithography was used to define an SU-8 mold for a 4-μL PCR chamber, 400 μm in depth and 3.6 mm in diameter. The mold incorporated two ports (650 and 660) with flow restrictions limiting the local vertical channel clearance to 20 μm, serving as passive weirs 680 to retain microbeads in the chamber, and a third port 690 with no weir to be used for bead insertion. Similar to the procedure above for fabricating the device depicted in FIG. 6a, the microdevice of 600′ was again fabricated from PDMS to form microchannel wall and supporting structure, which was bonded to a glass slide integrated with a resistive heater 630 and temperature sensor 640.

In addition to designing and fabricating the microchip, the bead-based primers were designed to allow rapid and simple operation of the device. Biotin-streptavidin coupling was used in this example to attach the reverse primers to the beads, as this bond is both strong and formed spontaneously in the presence of both molecules. The reverse primers were synthesized with a dual-biotin label at the 5′ end, followed by a spacer molecule adjacent to the nucleotide sequence. The dual-biotin moiety can minimize the loss of signal due to thermal denaturation. Spacer molecules provide greater lateral separation between DNA on the beads, thereby reducing hybridization issues due to steric hindrance. Synthetically generated template DNA was used to obtain controlled, consistent results for characterization of DNA detection using bead-based PCR.

The bead-based PCR chip was applied to pathogenic DNA detection, demonstrated with a DNA sequence associated with B. pertussis. B. pertussis is a gram-negative bacteria that infects ˜48.5 million patients (with 300,000 fatalities) annually worldwide. While early detection is the key to the treatment of this disease, current methods (e.g., cell culturing) for detecting the B. pertussis bacterium take days or even weeks of turnaround time. This limitation can be addressed by the disclosed bead-based PCR microchip which allows rapid, sensitive, and specific detection of B. pertussis. Pathogenic DNA detection using bead-based PCR on a microchip can be accomplished as follows: Bead-tethered reverse primers loaded in the microchamber of the microchip can be exposed to a raw sample, such as cell lysate, which can include various impurities. Pathogenic DNA in the sample can then be captured onto the beads via its specific hybridization to the reverse primers. As the capture is based on the affinity between the DNA and the reverse primers, this also serves as a purification step. Thereafter, the pathogenic DNA can be mixed on-chip with PCR reactants and bead-based PCR of the pathogenic DNA can be performed using fluorescently labeled forward primers. This process can rapidly generate exponentially amplified, fluorescently labeled template copies on microbeads, which can be detected by fluorescent microscopy. The use of beads in the detection volume can generate an enhanced signal-to-noise ratio in comparison to amplification on flat solid surfaces due to a significant increase in the fluorophore-coated surface area. Finally, the labeled copies of the template can be released from their bead-bound complements by denaturation and eluted into pure buffer for further analysis, while the bead-bound complementary strands can be retrieved from the chip and stored as cDNA libraries.

For detection of DNA associated with B. pertussis, the following materials and reagents were used. All DNA was obtained in lyophilized form from Integrated DNA Technologies, Coralville, Iowa, USA. The primers are designed as a PCR assay for determination of B. pertussis infection. The DNA sequences used are as follows—forward primer: 5′-FAM-Spacer-GAT TCA ATA GGT TOT ATG CAT GGT T-3′ (SEQ ID NO:1), reverse primer: 5′-Double Biotin-Spacer-TTC AGG CAC ACA AAC TTG ATG GGC G-3′ (SEQ ID NO:2), and template: 5′-GAT TCA ATA GGT TGT ATG CAT GGT TCA TCC GAA CCG GAT TTG AGA AAC TGG AAA TCG CCA ACC CCC CAG TTC ACT CAA GGA GCC CGG CCG GAT GAA CAC CCA TAA GCA TGC CCG ATT GAC CTT CCT ACG TCG ACT CGA AAT GOT CCA GCA ATT GAT CGC CCA TCA AGT TTG TGT GCC TGA A-3′ (SEQ ID NO:3). The forward primer has been modified with the fluorescent label carboxyfluorescein at the 5′ terminus, while the reverse primer incorporates a dual-biotin modification at the 5′ end. Both molecules contain an inert spacer molecule between the 5′ modifications and the nucleotide sequence. PCR was performed using Tag enzyme, deoxynucleotide triphosphates (dNTPs), and PCR reaction mixture containing appropriate buffers (Promega GoTaq Plexi PCR Mix). Reverse primers were immobilized onto streptavidin coated polymer-based microbeads (Thermo Scientific Pierce Protein Research Products Ultralink Streptavidin Resin) averaging 80 μm in diameter. Concentration and purity measurements of DNA samples were conducted using UV/VIS (Thermo Scientific Nanodrop). Materials used in microfabrication included photoresist (Rohm & Haas Electronic Materials S1818, Microchem SU-8 2000), PDMS prepolymer (Dow Corning Sylgard 184), and Parylene C prepolymer (Kisko diX C).

PCR reaction mixture for B. pertussis DNA detection was prepared as follows. Each lyophilized DNA sample was suspended in deionized H₂O and diluted to the desired concentration. The PCR mixture consisted of the following: 5×PCR Buffer (2 μL), 25 mM MgCl₂ (0.6 μL), 10 mM dNTPs (0.4 μL), 50 μg/mL BSA (0.4 μL), 5% (by volume) Tween 20 (0.1 μL), microbeads (0.5 μL), water (4.1 μL), 25 μM forward primer (0.4 μL), 25 μM reverse primer (0.4 μL), and enzyme (0.1 μL).

The ingredients were mixed with target (template) DNA (1 μL of synthetic template DNA with a concentration range of 1 aM-100 pM) without the enzyme and the mixture was then degassed at −0.4 psi for 30 min in a darkened container (to prevent photobleaching of the fluorophore label). Testing in the PCR device was also performed under an enclosure to prevent excess light from reaching the DNA. Following degassing, enzyme was added to the mixture and the 10 μL PCR sample was pipetted into the chip, followed by 30 μL of mineral oil. During the testing with the integrated device (as depicted in FIG. 6b and described above), PCR reaction mixture was degassed without primer-coated beads. These were inserted into the device, followed by template DNA. after which the DNA and beads were allowed to incubate for 10 min. This solution was then removed (with the beads retained by the weirs), and the PCR reaction mixture was introduced. The temperature of the sample was then cycled using the Labview control program to implement the reaction. For example, the reaction temperature was controlled by the Labview program that utilized sensor feedback to maintain a constant temperature field inside the chamber. A desktop power supply (Agilent E3631A) and a digital multimeter card (NI PCI-4060) provided electrical power and resistance measurements. An inverted fluorescence microscope (Nikon Diaphot 300) was used for all fluorescence measurements, while an attached digital camera (Pixelink PL-B742U) was used to record images of the excited fluorescent field. The microscope contains a dichroic mirror which attenuates light above the peak absorption wavelength of the fluorophore (˜494 nm) during excitation and passes the higher emission wavelengths (which peak at ˜512 nm) through the objective for observation and measurement.

Following PCR, the sample was pipetted to a darkened 0.5 mL microcentrifuge tube. Beads were washed six times with 1×SSC buffer to remove excess labeled primers. As used herein, the “x” before a buffer refers to the concentration, overall, as compared to the literature value of a standard buffer (which are prescribed in literature). For example, a 10×SSC buffer is ten times as concentrated as would commonly be used, and stored that way so as to allow the preparation of 1× solutions by adding additional water or desired reagents in solution (for example, 1 mL 10× buffer can be added to 9 mL of a DNA sample to achieve a 1× buffer solution with a desired concentration of DNA). Bead washing was by mixing the sample with buffer, allowing the beads to settle via gravity, and removing the supernatant with a pipette. A five microliter aliquot of each test sample was pipetted into an individual 3.2 mm diameter PDMS well on a glass slide, and was observed using the fluorescent microscope. During integrated device testing, microbeads were washed by passing buffer through the chamber while being retained by the weirs prior to fluorescent measurement. The microscope was kept in an enclosure to prevent ambient light from interfering with the measurements or bleaching the fluorescent labels. The samples containing the beads with attached ds-DNAs were briefly excited with light using the fluorescent light source and the resulting emission was recorded using the CCD camera microscope attachment. Camera exposure times were optimized based on fluorescent signal intensity during device characterization to maximize the signal-to-noise ratio, defined here as the ratio of the measured fluorescence intensity to the intensity of background fluorescence. Digital images were analyzed using Image J software.

The resistive heater and sensor were first characterized for accurate on-chip temperature control. For this test, the microchip was placed in a temperature-controlled environmental chamber and its temperature varied. Chamber temperatures were measured using a platinum resistance temperature detector probe (Hart Scientific 5628) and on-chip resistances were measured with a digital multimeter (Agilent 34420A).

Resistance measurements of temperature sensors indicated a linear relationship between resistance and temperature. These data were used to calculate a TCR for the sensor of 2×10⁻³° C.⁻¹. The heater was found to have a resistance of ˜20 Ω.

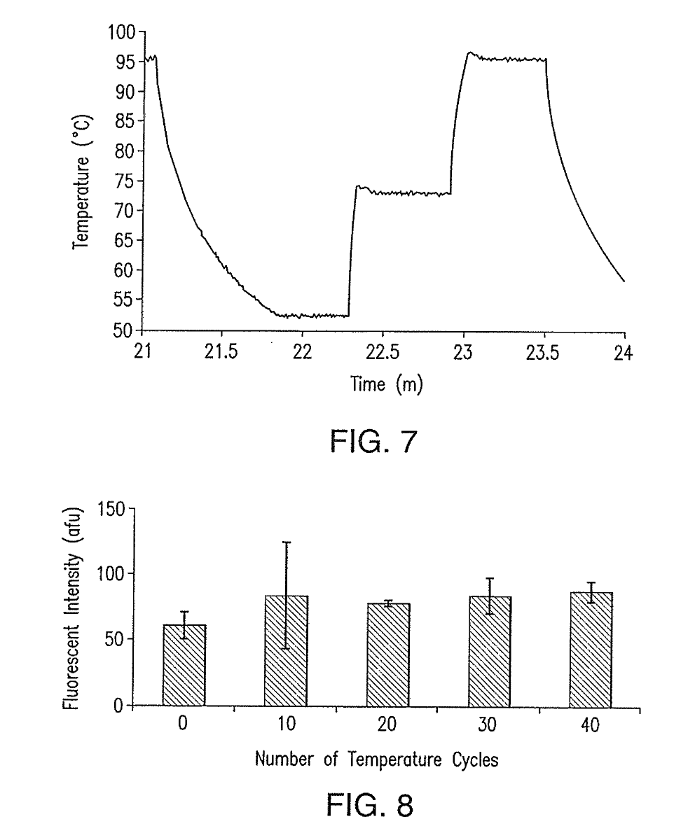

The accuracy of on-chip temperature measurements and the heating rate of the chip were then tested. A 1.5 mm diameter insulated K-type thermocouple probe (Omega Engineering) was inserted into the sample chamber along with a pure water sample. The chamber temperature was then controlled (heated cyclically) as would occur during a typical PCR test, but without the amplification reagents. According to the time course of temperature obtained during this control test (FIG. 7), the chip achieved target temperatures with minimal overshoot. The device exhibited an average time constant of heating (based on an exponential fit) of ˜1.4 s. In addition, the thermocouple readings agreed with the temperature setpoints to within ±0.5° C. This indicates that the chamber temperature can be effectively controlled to for the amplification reactions.

The effects of test conditions, such as ambient light and temperature, on test results were also investigated. Biotin-streptavidin binding was chosen as a simple alternative to covalent methods for DNA immobilization, however, streptavidin molecules can denature as a result of the elevated temperature necessary to dehybridize DNA. Temperature cycling equivalent to typical PCR testing was performed on linkages between streptavidin and dual-biotin labeled DNA. Beads coated with streptavidin were mixed with 1 μM dual-biotin labeled primers and an equal concentration of fluorophores-labeled complementary strands. The solution was subjected to temperature cycling, returned to room temperature, and washed to remove any DNA in solution. In FIG. 8, the fluorescent intensity is shown for untested beads (zero temperature cycles) and for beads subjected to 10, 20, 30, or 40 rounds of temperature cycling. (No PCR reagents were used in the cycles, thus no amplification products were produced in the procedure.) Intensity, measured in arbitrary fluorescence units (a.f.u. or afu), did not vary from baseline (zero temperature cycles) as a result of temperature cycling. This indicates that the concentration of DNA on the bead surfaces changes negligibly as a result of the PCR process, producing minimal error as a result.

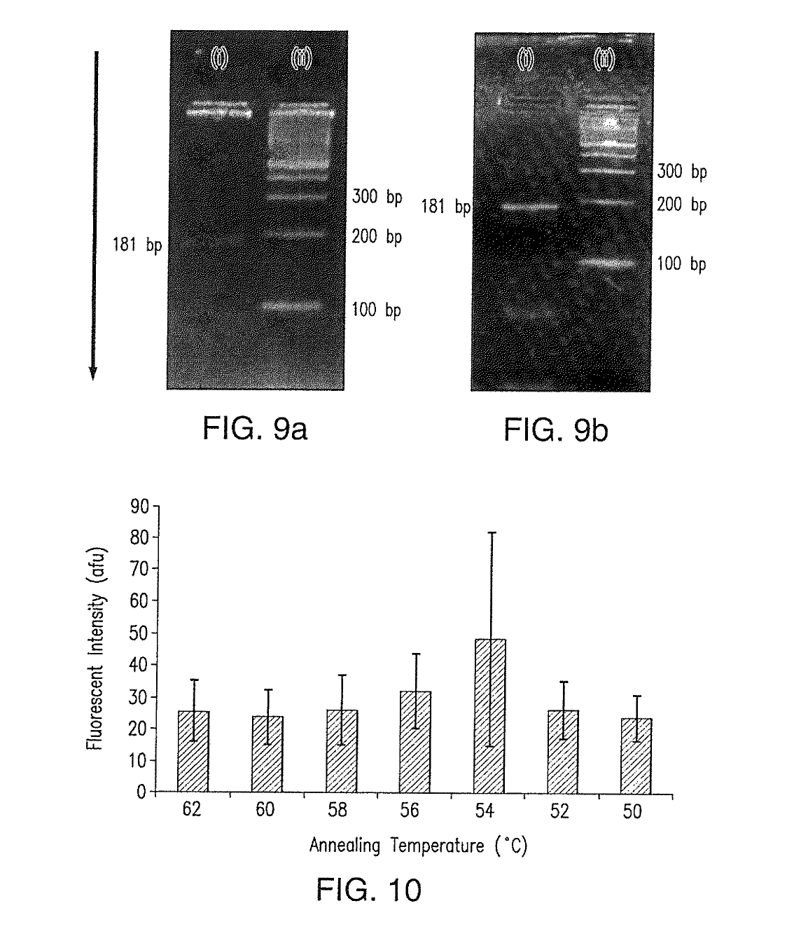

During a series of PCR reactions, parameters were varied individually and the results examined to determine the parameters that would generate maximum signal intensity, thus lowering the detection limit of the device. First, solution-based PCR (i.e., excluding microbeads) was performed to confirm device calibration and sequences of both DNA primers (25 bases) and template (181 bases). Following amplification, gel electrophoresis was performed and results indicate that a strand of the expected length (181 bp) was produced (FIG. 9a). This confirmed that the device temperature control is accurate enough to perform PCR, and that the DNA had been properly designed and synthesized. This test was then repeated using bead-based PCR and produced identical results (FIG. 9b), following DNA recovery from the beads via biotin-streptavidin denaturation in a 95° C. formamide bath. Following ethanol precipitation of the DNA and resuspension in distilled H₂O, gel electrophoresis was performed. The results indicate that coupling the reverse primers to the beads does not cause improper DNA amplification (such as the generation of spurious products). In addition to the comparison with solution-based PCR, bead-based PCR tests were conducted with smaller reaction volumes (5 μL), and this was not found to influence the average fluorescent signal intensity following amplification.

An exemplary magnesium concentration was determined to be 1.5 mM, consistent with typical MgCl₂ concentrations for PCR studies. A series of tests also determined that a consistent 20 s dwell time, or time spent at each temperature setpoint during a PCR cycle, would produce DNA most efficiently for the microdevice employed.

The effect of annealing temperature on the concentration and length of DNA generated during PCR was investigated. The annealing temperature can affect the hybridization of the primers to the template DNA; a higher annealing temperature can result in more specific hybridization (less erroneous hybridization of a primer to an unspecified DNA sequence), but less total hybridization of DNA (and therefore less product DNA after PCR). A series of bead-based PCR tests was conducted using the B. pertussis primer set in which the annealing temperature was varied. The results indicated that fluorescent intensity of the beads following PCR remained approximately consistent (FIG. 10). Large variation in individual test results was observed for tests with an annealing temperature of 54° C.; nonspecific annealing at this temperature can cause variability in reaction efficiency.

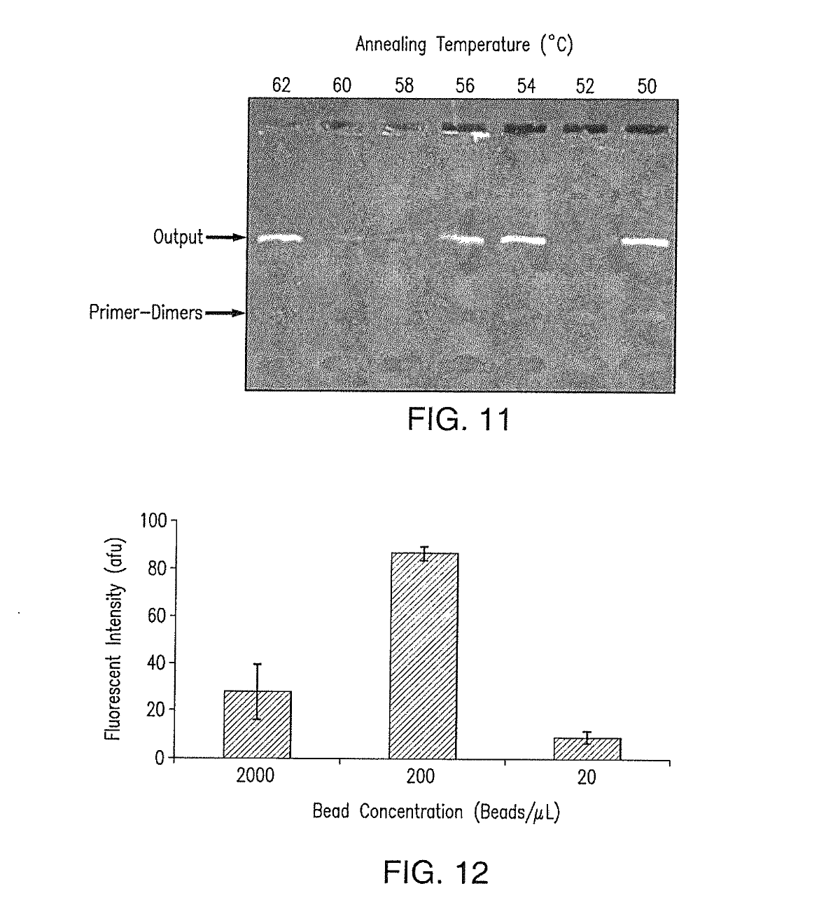

The annealing temperature test was then repeated with conventional solution-based PCR, and the results were analyzed using gel electrophoresis (FIG. 11). Results indicated no amplification at 52° C., very close to the temperature at which a high degree of variability was observed in the bead-based tests. As it appears that this range of annealing temperatures produces sporadic results, a higher annealing temperature was chosen for further testing. The results from the solution-based test that an annealing temperature of 58° C. minimized generation of primer-dimers, a type of non-specific amplification which occurs when two primers hybridize and are extended during PCR. Bead-based PCR results cannot discern the length of generated DNA, so non-specific amplification represents a source of false positive readings during DNA detection. This effect should be inhibited to improve detecting specificity, Example PCR cycle parameters (cycle times and temperatures) after the above investigations are summarized in Table 1 below.

TABLE 1
Summary of PCR cycle parameters

| | Temperature | Dwell |
|---|---|---|
| Stage | (° C.) | time (s) |
| Pre-melting | 95 | 60 |
| Thermal cycling | 95 | 20 |
| | 58 | 20 |
| | 72 | 20 |
| Post-extension | 72 | 180 |

Next, the bead-based PCR detection device was optimized with respect to the concentration of beads in the reaction mixture. The presence of a solid surface in bead-based PCR introduces steric and geometric effects, affecting the reaction efficiency. Previous studies of solid-phase amplification focused on maximizing the final concentration of DNA, however, the primary concern of this study is the detection of DNA. The concentration of microbeads in the reaction mixture was therefore investigated and optimized to produce the greatest fluorescent signal. To test the effects of bead concentration on fluorescent signal intensity, bead-based PCR reactions were performed using conditions (58° C. annealing temperature, 1.5 mM MgCl₂ concentration, 10 pM template concentration) and three different concentrations of beads (FIG. 12). It can be seen that ˜200 beads/μL generates the most intense fluorescent signal on the beads, with a significant decrease in signal intensity at either a lower or a higher concentration.

The sharp peak in FIG. 12 can be explained by the relationship between the concentration of microbeads in the reaction mixture and the total surface area of the surface-based PCR reaction. At higher concentrations of microbeads, the fluorescently labeled product DNA is spread across a larger number of beads. The greater surface area results in a weaker fluorescent signal because the signal strength is proportional to the surface density of the fluorescent labels. On the other hand, a lower concentration of beads does not imply a higher surface concentration of DNA. At these bead concentrations, the greater density of reverse primers limits the reaction by steric hindrance between molecules at the bead surfaces. Increasing proximity of reverse primers to one another on solid surfaces can hinder the ability of DNA in solution to hybridize to the bead-bound primers. In addition to steric hindrance, the increasing density of the fluorophores on the bead surfaces can result in quenching due to proximity of other fluorophores and nucleotides, which can inhibit fluorescence. Alternatively, a lack of microbeads in the reaction can cause some reverse primers to remain in solution, as each bead can support a limited number of primers. The competition of these in-solution primers for the template DNA and the lower efficiency due to steric hindrance account for the drop in signal intensity at lower bead concentrations. FIG. 12 shows that there is a 9-fold decrease in signal intensity from 200 to 20 beads/μL, indicating that bead concentration strongly influences the ability of the sensor to detect DNA.

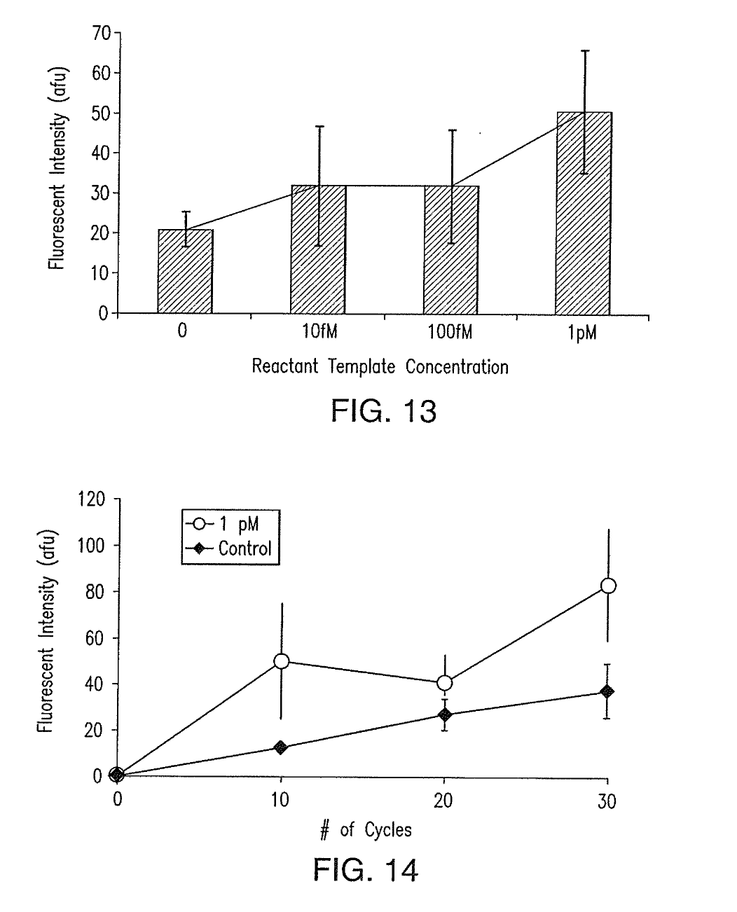

The microdevice was then tested for detecting synthetic gDNA. Detection criteria included the limit of detection (the smallest concentration of DNA that could be detected) and the number of PCR cycles necessary for detection. The limit of detection of the device was investigated by performing a series of PCR reactions while changing the concentration of template DNA in the reactants. Concentrations ranging from zero templates to 1 pM (˜6×10⁵ copies/μL) were tested. PCR reactions were run for 10 cycles (˜15 min) using the optimized test conditions discussed above (200 beads per microliter, 20 s dwell times, 58° C. annealing temperature, and 1.5 mM MgCl₂). As seen in FIG. 13, template DNA concentrations of 0.1 pM or less produced an increase in fluorescence above the zero template reaction. Zero template amplifications produced a fluorescent signal because non-specific amplification products such as primer-dimmers were detected in addition to properly amplified DNA. This effect was investigated further in the following discussion of signal intensity versus cycle number. A template concentration of 1 pM, however, produced a signal distinguishable from the zero template control, as determined by the Student's t test with a 95% confidence level. This detection limit is orders of magnitude smaller than limits when using PCR with electrochemical labels or optical detection on a flat surface.

In addition to measuring the detection limit of the device, the relationship between signal intensity and the number of PCR cycles performed prior to detection was also investigated. For this investigation, bead-based PCR reactions were performed as discussed in the previous section, but a 1 pM (˜6×10⁵ copies/μL) concentration was consistently used and the number of PCR cycles was varied. As seen in FIG. 14, signal intensity at 10 cycles is well above background levels and increases from 10 to 30 cycles. There is a decrease in signal from 10 to 20 cycles. A number of detrimental effects are can occur, such as non-specific amplification between two surface-bound primers or creation of sterile (incomplete) molecules, both of which could then reverse themselves with further amplification. In addition to amplification of a 1 pM sample, a control sample with no template DNA was also amplified and the results are shown in FIG. 14. The control signal shows a linear increase in intensity, but consistently remains below that of the test sample.

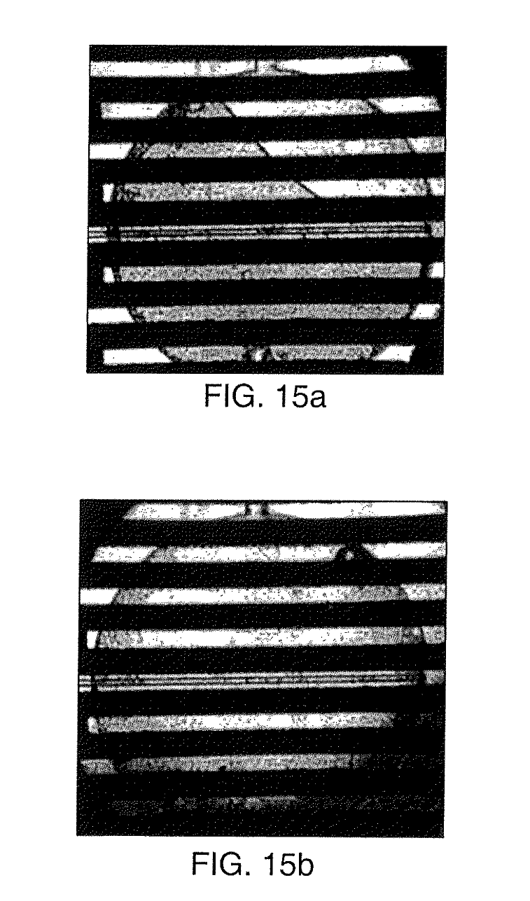

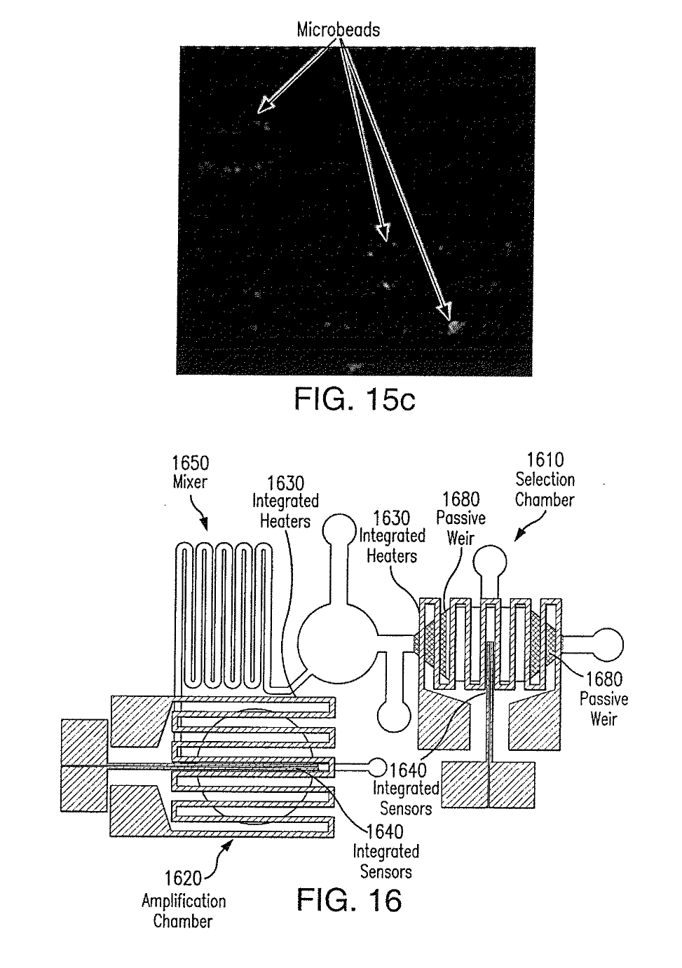

FIGS. 15a-15c show micrographs of the microchamber illustrating the process of integrated microfluidic bead-based PCR. In FIG. 15a, a brightfield micrograph shows a microchamber in which beads have been inserted into the chamber and a DNA solution is being introduced (fluid flowing from top to bottom). In FIG. 15b, the DNA solution has been introduced, and template DNA is being captured from the solution onto bead surfaces by bead-bound reverse primers. In FIG. 15c, following PCR cycling, and washing with buffer, the fluorescent intensity of the microbeads was measured. A 5 μL volume of microbeads (concentration ˜200 beads/μL) was inserted into the microchamber (shown in FIG. 15a), and 10 pM template DNA (˜6×10⁶ copies/4) was then inserted and allowed to incubate for a period of 10 min (FIG. 15b). This solution was then removed and PCR reaction mixture introduced, at which point the integrated heaters and sensors were used to cycle the chamber temperature to effect 10 cycles of PCR. Following amplification, the reaction mixture was removed by washing with pure buffer, leaving only beads coated with fluorescently labeled DNA in the microchamber (FIG. 15c). Analysis of the fluorescent micrograph indicated that the beads fluoresced with an intensity of 75.9 afu. The chamber temperature was then raised and maintained at 95° C. for 5 min, denaturing the bead-bound ssDNA and eluting it into a pure buffer solution. UV/VIS spectroscopy confirmed that the eluent contained 163.6 ng/μL ssDNA. A fluorescent micrograph of the microbeads that remained in the microchamber (not shown) confirmed that product ssDNA had indeed been removed from the beads. These results illustrate that the use of bead-based PCR can enable the design of a highly integrated microchip for DNA purification and detection, with precise control of buffer conditions during on-chip procedures.

### Example 2

In this example, aptamers are selected and amplified using an integrated microchip including a selection chamber and an amplification chamber, as illustrated in FIG. 2, as previously described. Briefly, binding sequences are isolated from a random library (FIGS. 2a-2c), chemically amplified via PCR (FIG. 2d), and single strands are then collected (FIG. 2e). The collected strands can be loaded into the selection chamber so that the procedure can be repeated.

The microdevice (or microchip, chip) used in this Example includes two chambers, one of which (selection chamber) performs selection and separation of candidate aptamers, and the other (amplification chamber) amplifies and collects the candidate aptamers. A schematic diagram of the microdevice for this Example is shown in FIG. 16, where the selection chamber 1610 is a 400 μm tall cuboid, with two inlet/outlets restricted to a height of 10 μm and one 400 μm tall inlet for insertion of microbeads. The amplification chamber 1620 is a 4 μl, cylinder 400 μm tall with two inlet/outlets. One such inlet allows for insertion of PCR reactants, including microbeads, while the other is 10 μm tall so that microbeads can be retained while the supernatant solution is removed. The use of bead retention structures (weirs 1680) in the microchip allows for precise control of buffer conditions during each step of the isolation process, by retaining desired nucleic acids on the microbeads while the solution is changed. Between the two chambers is a serpentine channel (mixer 1650) which serves to mix ssDNA from the selection chamber with PCR reagents via diffusion. Resistive heaters 1630 and sensors 1640 are placed directly beneath each microchamber to control chamber temperature. The inner chamber and channel surface is coated with Parylene C to minimize adsorption of reactants and vapor losses.

All DNA used in the tests in this Example was purchased from Integrated DNA Technologies (IDT), with sequences as follows. Library: 5′-CTA CCT ACG ATC TGA CTA GCN NNN NNN NNN NNN NNN NNN NNN NNN NNN NNN NNN NNN NNN GCT TAC TCT CAT GTA GTT CC-3r (SEQ ID NO:4). Forward primer: 5′-FAM-Spacer-C TAC CTA CGA TCT GAC TAG C-3′ (SEQ ID NO:5). Reverse primer: 5′-Dual Biotin-Spacer-G GAA CTA CAT GAG AGT AAG C-3′ (SEQ ID NO:6). Library and primer sequences are based on a conventional SELEX protocol targeting IgE. Reagents used for PCR include 5× GoTaq Flexi PCR mix (Promega), 25 mM MgCl₂, 10 mM dNTPs (Promega), 50 μg/mL BSA (Sigma), GoTaq enzyme (Promega), and streptavidin-coated polymer microbeads (Streptavidin Plus Ultralink, Pierce). Beads used in the selection procedure were Bio-Rad Affi 10 Gel activated media with 80 μm average diameters. The functional molecules used to capture the target DNA (also referred to as target protein) used was human IgE (Athens Research).

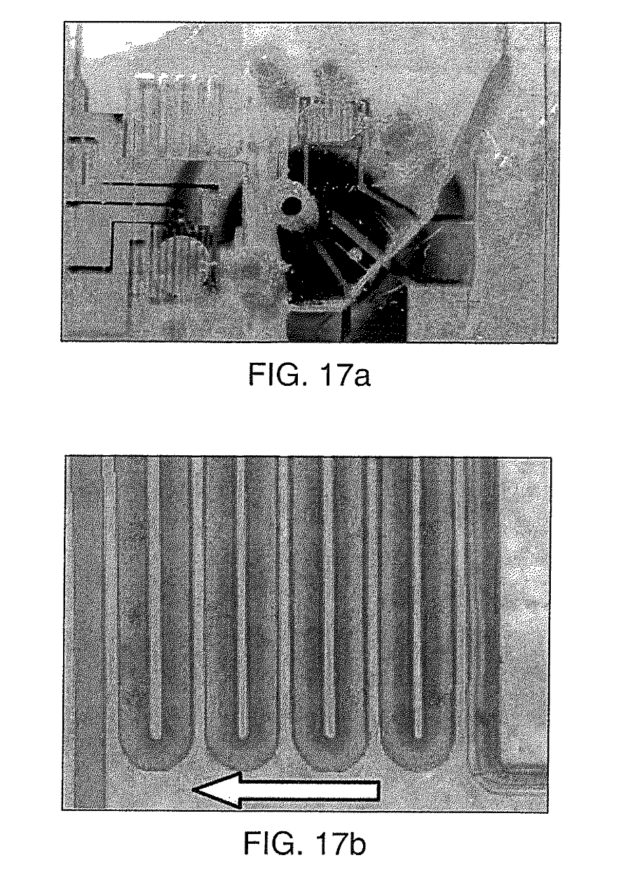

The microdevice was fabricated using contact lithography. Briefly, glass slides were coated with chrome and gold (15 nm and 150 nm thick, respectively), patterned using optical lithography, and etched to produce resistive heaters and temperature sensors. A 1 μm film of silicon dioxide was then applied via a plasma-enhanced chemical vapor deposition (PECVD), with a silicon hard mask defining openings for electrical connections. Molds for soft lithography were defined using optical lithography of layered SU-8 on silicon wafers. PDMS was then cast to produce microfluidic channels, chambers, weirs, and the mixer. The PDMS fluidic network was then bonded to the glass slide following oxygen plasma treatment, and the entire chip was coated with 1 μm of Parylene C via CVD prior to packaging (as shown in FIG. 17a).

Following fabrication, the resistive temperature sensors were calibrated in an environmental chamber and the mixer was tested using a dye to measure mixing efficiency (as shown in FIG. 17b). Prior to testing, IgE-coated heads were loaded into the selection chamber until fully packed, and the bead inlet was sealed with wax. Beads were then washed once with 1× phosphate buffered saline (PBS) prior to testing, and the entire chip was exposed to a 50 μg/mL BSA solution to prevent non-specific adsorption of DNA. The chamber temperature was then set to the desired selection temperature (T₁), 37° C., and three 30 μL aliquots of 10 μM library DNA solution in 1×PBS modified with 1 mM MgCl₂ (PBSM) was introduced to the selection chamber at 5 μL/min. Following exposure to the library, the chamber was then rinsed with ten 30 μL aliquots of 1×PBSM, also at 37° C., to remove unbound or weakly bound ssDNA. The selection chamber was then set to the elution temperature (T₂), 57° C., and after 5 minutes of incubation, four 30 μL aliquots of modified PBS were inserted at 5 μL/min to elute candidate aptamers.

Buffer containing candidate aptamers was mixed with PCR reagents and microbeads, and introduced to the amplification chamber. Remaining unamplified solution was separately removed from the chip and stored. With the amplification chamber filled, the inlets were sealed with wax and the solution thermally cycled. Fluorescence intensity of the beads confirms final surface concentration of DNA. The chamber was then held at 95° C. for 5 minutes to dehybridize bead-bound DNA, and buffer was then flowed at 1 μL/min to remove amplified ssDNA.

Binding analyses were performed in the selection chamber using a fresh chip containing fresh IgE-coated beads. A sample of enriched library DNA was further amplified off-chip, again using FAM-labeled forward primers. This was then purified with streptavidin-coated microbeads; ssDNA was eluted at 95° C., and resuspended in 1×PBSM. Sample concentration was then measured with UV/VIS absorption (ThermoScientific Nanodrop) prior to further analyses.

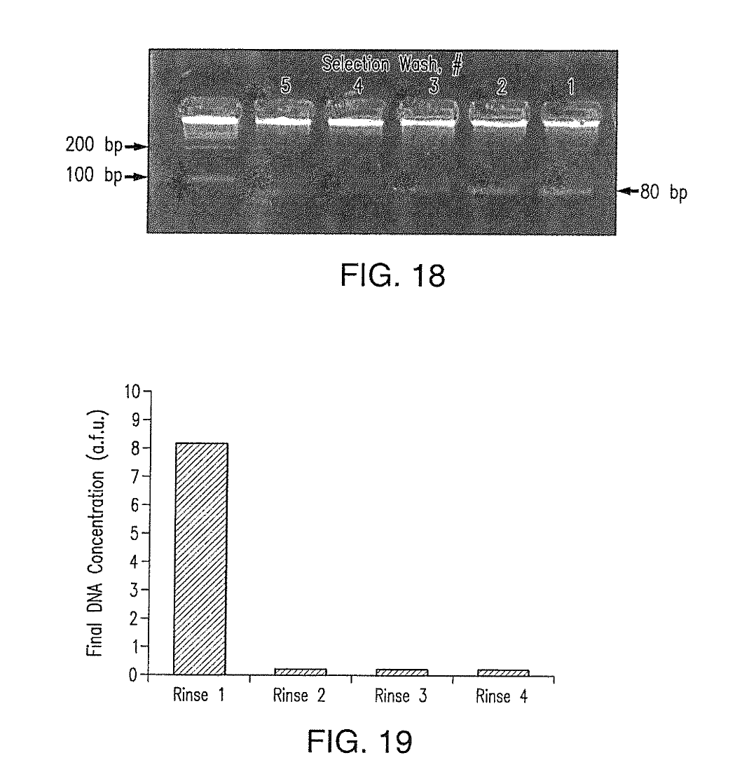

A portion of each buffer sample used for washing the beads in the selection chamber was removed and stored for testing following isolation and amplification. These samples were analyzed off-chip using conventional PCR, in parallel with on-chip amplification and collection. To measure the effect of on-chip aptamer isolation on the randomized pool, each of ten buffer aliquots which washed the IgE-coated beads at 37° C. was amplified off-chip and tested using gel electrophoresis. This generated a clearly defined gradient of concentration, with bands corresponding to initial washes fluorescing brighter than those corresponding to the following washes (FIG. 18). This indicates that as selection proceeded, fewer weakly bound strands of library DNA were being removed from the bead-bound IgE in the selection chamber, increasing the selection stringency. Amplification of the washes at 57° C. provided a similar measure of the effects of selection. By amplifying each sample of eluted aptamer candidates, the presence of candidates and the efficiency of the elution protocol are both illustrated. Following the isolation washes at 37° C., ssDNA was eluted at 57° C., mixed with PCR reagents on-chip, and subjected to 17 cycles of PCR amplification. Measurements of the fluorescent intensity of microbeads following on-chip bead-based PCR of four aliquots of aptamer candidates showed a discrete change from a strong signal following amplification of the first sample, to a minimal signal in ensuing tests (FIG. 19). This sudden change indicated that the desired aptamer candidates were almost entirely eluted in the first wash at 57° C.

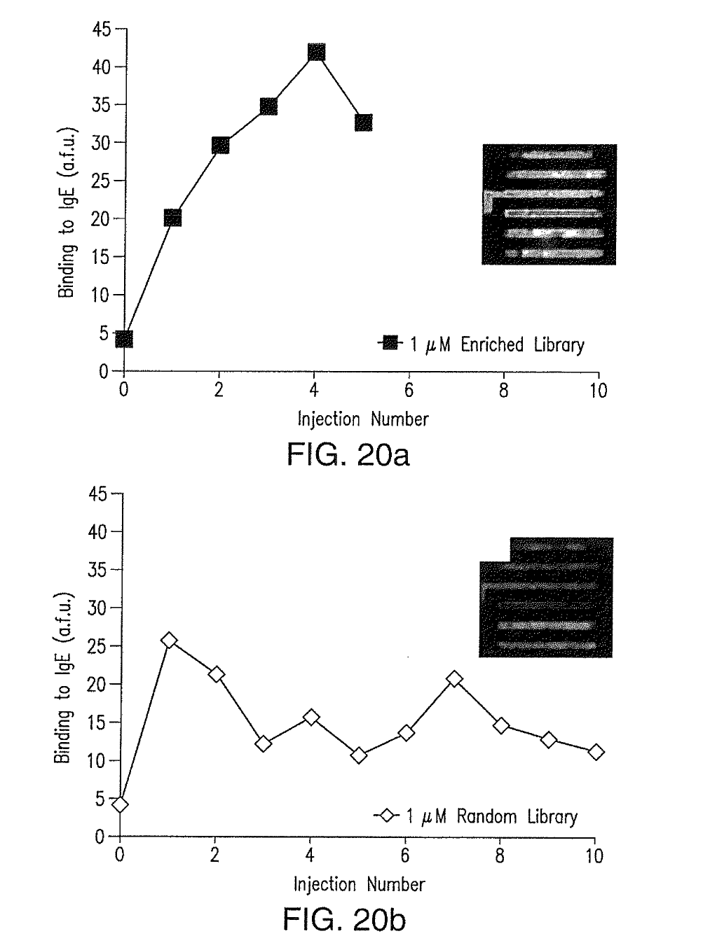

Further, the affinity of the enriched pool of aptamer candidates for IgE was tested. To generate a quantity of DNA large enough for testing, the enriched pool was amplified off-chip using conventional PCR. Following isolation and resuspension of the target ssDNA in 1×PBSM, the concentration of the sample was measured with UV/VIS and normalized to 1 μM. For comparison, randomized library was purchased containing a FAM modification at the 5′ terminus, and diluted to 1 μM in 1×PBSM. Samples of each (5 μL) were individually inserted into a microchamber containing microbeads freshly coated with IgE and maintained at 37° C., incubated for 5 minutes, washed with buffer, and micrographed during fluorescent excitation. The increased affinity of the enriched pool versus the randomized library was easily viewable using an optical microscope (FIG. 20). Analysis of fluorescent intensity of micrographs after incubation of each 5 μL aliquot confirms that the enriched aptamer pool had much higher affinity for IgE.

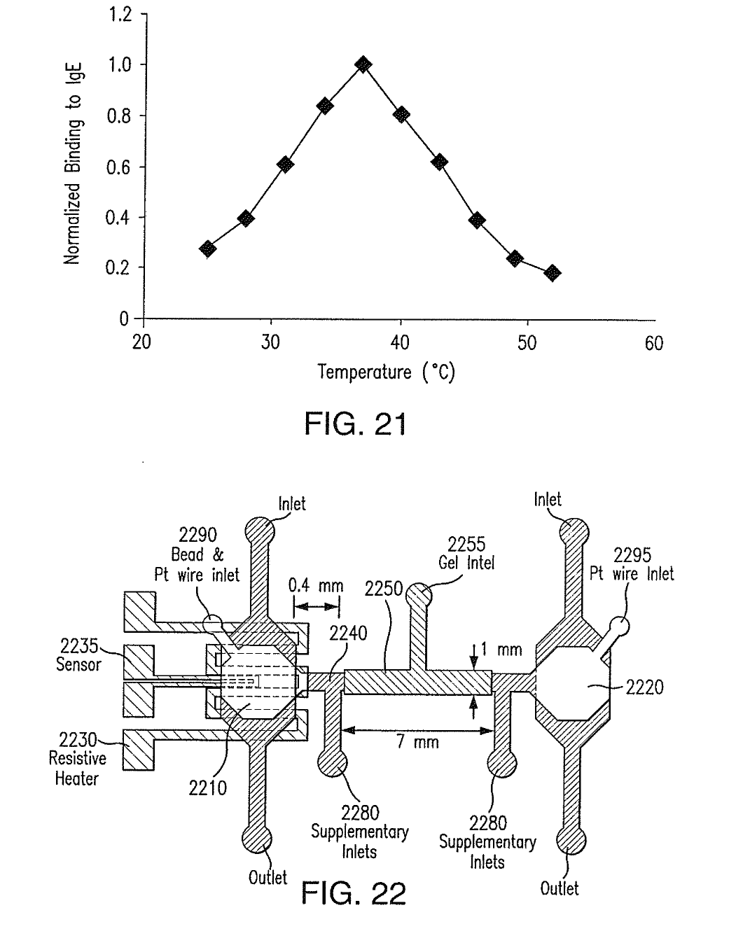

In addition to measuring the increased affinity of the enriched pool for IgE, the temperature-dependence of the affinity was measured. As with the affinity measurements, a 1 μM solution of fluorescently-labeled aptamer candidates in 1×PBSM was exposed to a chamber packed with microbeads coated with IgE until the fluorescent signal saturated. Pure buffer was then flowed at 1 μL/min, while the temperature of the chamber was changed in increments of 3° C. While buffer was continuously flowed, the chamber was maintained at each temperature for 5 minutes. The pool exhibited highly temperature-dependent binding to IgE, with maximum binding at 37° C. as desired (FIG. 21). These temperature-sensitive aptamer candidates can be isolated much faster and more efficiently than with conventional technology (see Table 2 below).

TABLE 2
Comparison of integrated microfluidic aptamer
isolation to conventional methods

| | Microfluidic | Conventional |
|---|---|---|
| | Isolation | Isolation |
| Sample Volume | 30 μL | ~250 μL |
| Assay Time | 4 hours | ~2 days |

### Example 3

In this example, isolation and enrichment of target DNA is demonstrated using a microfluidic chip having two chambers and a channel therebetween which includes a gel, as previously described in connection with FIG. 3.

As shown in FIG. 22, the microchip used in this Example includes two microchambers, the selection/isolation microchamber 2210 and the enrichment microchamber 2220, each having a depth of 200 μm, and volume of 5 μL, connected by a microchannel 2240 (length: 7 mm, width: 1 mm, height: 300 μm). A weir structure (height: 40 μm) in the isolation chamber retains microbeads (diameter: 100 μm) in that chamber during the isolation and enrichment processes. The resistive heater 2230 and temperature sensor 2235 integrated on the glass substrate were used to manipulate the temperature in the isolation chamber during the thermal elution of ssDNA from the beads. The connecting channel 2240 is partially filled with agarose gel 2250 through an inlet 2255. An additional length of the channel (length: 0.6 mm, width: 0.4 mm, height: 40 μm) thermally insulates the solidified gel from the heated chambers during the thermal elution. Supplementary inlets 2280 were used to fill these additional channel areas with buffer. An electric field was formed across the microchannel by a potential difference applied via Pt wire electrodes that are inserted into the microchambers through the Pt wire inlets 2290 and 2295.

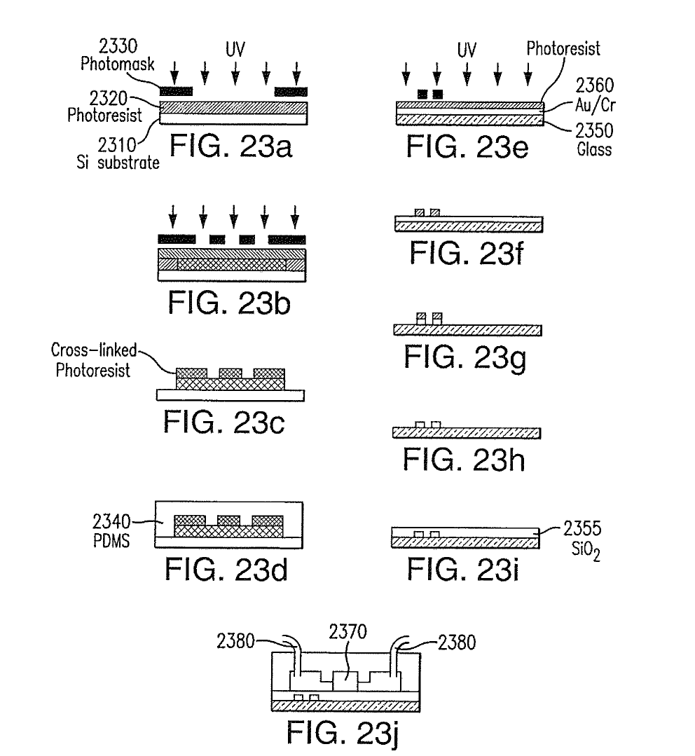

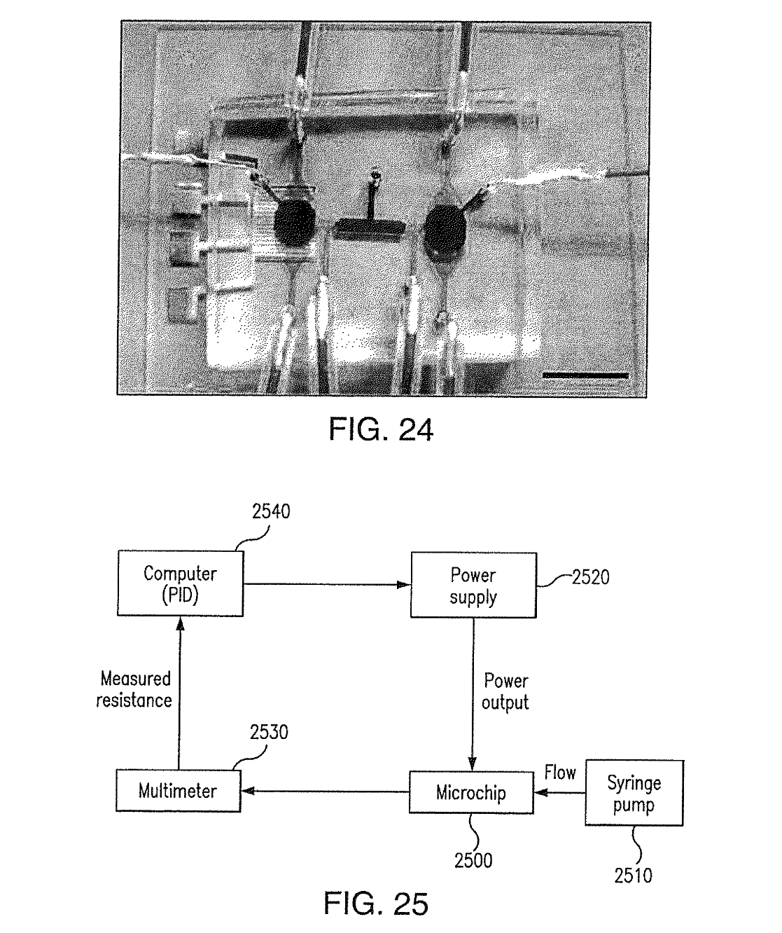

The microchip used in this Example was fabricated from a polydimethylsiloxane (PDMS) microfluidic layer bonded onto a glass substrate patterned with a resistive heater and sensor using standard microfabrication techniques such as lithography (FIG. 23). To prepare an SU-8 mold for the PDMS layer, a silicon wafer was cleaned by soaking in piranha solution (a mixture of 98% sulfuric acid and 30% hydrogen peroxide, 3:1, v/v) for 1 hour. The wafer was then rinsed in deionized water and baked on a hotplate at 180° C. for 15 minutes. Layers of SU-8 photoresist 2320 were spin-coated on the silicon wafer 2310 and exposed to ultraviolet light through photomasks 2330, and baked to define a mold (FIGS. 23a-23c). PDMS pre-polymer (Sylgard 184, Dow Corning) was then spread onto the SU-8 mold, baked at 75° C. for 1 hour on a hotplate, and peeled off from the mold thereby forming the wall 2340 of the microchamber and channel (FIG. 23d). Separately, chrome (thickness: 5 nm) and gold (thickness: 100 nm) layers 2360 were consecutively deposited on a piranha cleaned glass substrate 2350 using a thermal evaporator (Auto 306, BOC Edwards). After the metal layers were patterned using positive photolithography (FIGS. 23e-23h), they were passivated with a silicon dioxide layer 2355 (thickness: 1 μm) using plasma-enhanced chemical vapor deposition (FIG. 231). After punching access holes for inlets and outlets in the PDMS 2340, it was bonded to the glass substrate 2350 following oxygen plasma treatment of the bonding surfaces. Inlet and outlet ports were connected to plastic tubes 2380 for sample handling. Molten agarose gel 2370 was injected using a micropipette to fill the microchannel through the gel inlet and was allowed to solidify (FIG. 23j). An image of the fabricated microchip is shown in FIG. 24.

To prepare IgE-functionalized microbeads, 200 μL of solution containing NHS activated microbeads (mean diameter: ˜100 μm, GE Healthcare) was washed 3 times with 1×PBS buffer modified to contain 1 mM of Mg²⁺ ions (8.1 mM Na₂HPO₄, 1.1 mM Na₂HPO₄, 138 mM NaCl, 2.7 mM KCl, 1 mM MgCl₂, pH 7.4) by centrifugation. Then the beads were incubated with 200 μL of 0.1 μM human myeloma IgE (Athens Research & Technology) for 5 hours at room temperature. After incubation, excess IgE molecules were discarded by washing the beads with fresh PBS buffer. To reduce nonspecific binding of ssDNA molecules to the beads, the surfaces that were not conjugated with IgE were passivated by incubating the beads in 0.1 M Tris-HCl buffer for 1 hour. The IgE-functionalized beads were stored in PBS buffer at 4° C. before use. An fluorescently labeled ssDNA library having random sequences (97-mer, TGT TGT GAG CCT CCT GTC GAA—50 random bases—TTG AGC GTT TAT TCT TGT CTC CC-3′) (SEQ ID NO:7), IgE-specific ssDNA aptamer D17.4 (78-mer, K_{D}=10 nM, 5′-GCC TGT TGT GAG CCT CCT GTC GAA GCA CGT TTA TCC GTC CCT CCT AGT GGC GTG CTT GAG CGT TTA TTC TTG TCT CCC-3′) (SEQ ID NO:8), and forward (5′-GCC TGT TGT GAG CCT CCT GTC GAA-3′) (SEQ ID NO:9) and reverse (5′-GGG AGA CAA GAA TAA ACG CTC AA-3′) (SEQ ID NO:10) primers were purchased from Integrated DNA Technologies. A mixture of random ssDNA and aptamer D17.4 (1000:1, mole ratio) was used throughout the example to increase competition for IgE binding sites. The random ssDNA solution was prepared by mixing 1 μL of a 100 μM random ssDNA library and 1 μL of 0.1 μM aptamer D17.4 in 98 μL of 1×PBS buffer. The running buffer for electrophoretic transport of ssDNA in the microchannel and for a slab-gel electrophoresis was 0.5×TBE buffer (44.5 mM Tris base, 44.5 mM boric acid, 1.25 mM EDTA, pH 8.3). Three percent agarose gel (Difco Laboratories) for electrophoresis was prepared by dissolving 0.3 grams of agarose in 100 mL of 0.5×TBE buffer on a hotplate.

A schematic of the test setup is shown in FIG. 25. The sample solutions including the ssDNA mixture and buffers were introduced into the microchambers using a syringe pump 2510 (NE 300, Harvard Apparatus). The temperature in the isolation chamber during the thermal elution process was maintained at 57° C. via the resistive heater and sensor connected with a power supply 2520 (E3631A, Agilent Technologies) and a multimeter 2530 (34410A, Agilent Technologies), respectively, that are controlled by a LabVIEW-based PID module 2540 on a computer. The Pt electrodes were connected to the power supply to apply a potential difference between the two chambers to induce electrophoretic transport of ssDNA strands. The transport of ssDNA through the gel-filled channel was monitored at the center of the channel using a fluorescence microscope (LSM 510, Zeiss).

Isolation and enrichment of desired ssDNA molecules in the randomized ssDNA mixture was carried out as follows. The IgE-functionalized microbeads were loaded in the isolation chamber using a syringe through a bead inlet to fill approximately 30% of the chamber volume (˜3×10⁴ beads). After loading, the beads were washed for 5 minutes with 1×PBS buffer at a flow rate of 40 μL/min using a syringe pump. The random ssDNA mixture (100 μL) was introduced to the chamber through the inlet at a flow rate of 20 μL/min and collected from the outlet in 3 separate plastic tubes (˜33 μL/tube). PBS buffer was injected to the chamber at 40 μL/min to wash weakly bound DNA strands from the IgE-beads, and the waste solution was collected in 10 separate tubes at the outlet (˜33 μL/tube). The two chambers were filled with 0.5×TBE buffer and then the isolation chamber was heated at 57° C. for 5 minutes via the resistive heater to elute strongly bound DNA strands from the beads.

As the thermal elution was occurring, Pt-wire electrodes were inserted into the chambers and an electric field of 25 V/cm was applied for 25 minutes. The DNA strands were then electrophoretically transported to the enrichment chamber through the gel-filled channel. To investigate the single round of isolation and enrichment of ssDNA, the two chambers were flushed with PBS buffer as eluents were collected in plastic tubes (˜33 μL/tube). For multiple rounds of DNA enrichment, the beads in the isolation chamber were discarded and the chamber was thoroughly washed with PBS buffer prior to the next round of the isolation and enrichment processes to remove undesired DNA molecules that might remain. Fresh IgE-functionalized beads were then introduced in the isolation chamber for the next round of DNA isolation and enrichment.

To analyze the results from the example, representative eluent samples from each step were amplified off-chip by polymerase chain reaction (PCR) using a thermal cycler (Mastercycler Personal, Eppendorf). The PCR procedure included denaturation of DNA at 95° C. for 3 minutes followed by 20 cycles of amplification. Each cycle consisted of denaturation at 95° C. for 15 seconds, annealing at 59° C. for 30 seconds, and extension at 72° C. for 45 seconds. Following the amplification, 7 μL of PCR product was mixed with 7 μL of 2×DNA loading dye containing bromophenol blue and xylene cyanol (Thermo Scientific) and loaded into each lane of a 3% agarose gel. Electrophoresis was then carried out at 100 V for 30 minutes in 0.5×TBE buffer using a slab gel apparatus (Mupid-exU, Advance). The gel was then stained with ethidium bromide in deionized water for 5 minutes. The bands in the gel representing the concentration of DNA in each eluent sample were visualized using a UV illuminator (AlphaImager 3400, Alpha Innotech). A fluorescence microscope was used to monitor the electrophoretic transport of ssDNA through the gel-filled channel. The intensities of gel-bands and fluorescence from images obtained were analyzed using the Image J software (National Institutes of Health freeware).

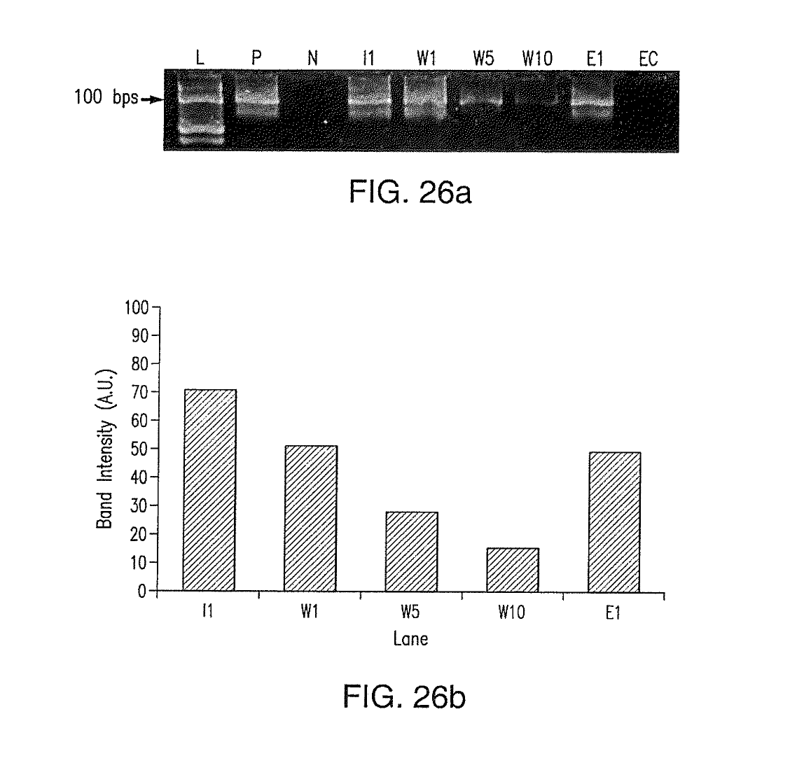

In the isolation-enrichment procedure, the IgE-binding ssDNA was first isolated from the randomized ssDNA mixture in the isolation chamber. IgE isolation was effected by exposing the chamber to samples of randomized DNA and then washing with pure buffer to remove unbound DNA. These buffer samples containing residual DNA were collected following washing, and were amplified with PCR and visualized with slab gel electrophoresis to determine the effectiveness of the isolation procedure. FIG. 26a shows a gel electropherogram of the PCR products of eluents collected during the isolation process. In the gel image, bands in lanes L, P, and N represent a 10 base pair (bp) DNA ladder, positive control (a PCR reaction in which template DNA consisted of 100 pmole random ssDNA and 0.1 pmole D17.4 aptamer) and negative control (a PCR reaction excluding template DNA), respectively. Additional bands represent amplified samples of eluent collected during incubation (lane I1), washing (lanes W1-W10), elution (lane E1), and buffer used to wash the enrichment chamber after the ssDNA isolation process (lane EC). Note that the numbers after the abbreviations of each process represent the order in which eluent samples were collected. For example, “5” in “W5” means the 5th eluent sample collected during the washing step.

The upper and lower bands seen in lanes P and I1-E1 represent amplified samples of the 97 bp random ssDNA and 78 bp D17.4 aptamer, respectively. The upper bands are brighter than the lower bands as a result of the 1000:1 molar ratio of random ssDNA to D17.4 aptamer in the DNA mixture used for the isolation. No bands are seen in lane N, indicating that the reagents used were not contaminated by undesired DNA molecules. In addition, no bands are seen in lane EC, indicating that the gel-filled microchannel effectively prevented contamination of the enrichment chamber with unwanted ssDNA from the isolation chamber during the capture of the target DNA strands.

A bar graph depicting the band intensity of the 97-mer random ssDNA (the upper band) from lane I1 to lane E1 is plotted to show the progress of the isolation of IgE-binding ssDNA (FIG. 26b). The DNA that did not bind to the IgE-coated microbeads during the incubation step is indicated by the high band intensity in lane I1. The decreasing intensity of the bands from lanes W1 to W10 indicates that as washing continued, loosely bound ssDNA molecules were removed from the bead surfaces, increasing the stringency of the isolation of target ssDNA. The increased band intensity in lane E1 indicates that strongly bound ssDNA molecules were eluted from the bead surface by heating at 57° C. In addition, no damage was observed to the agarose gel in the electrophoresis channel, indicating that the channel length between the microchamber and the gel-filled channel was large enough to prevent thermal degradation of the gel during elution of DNA.

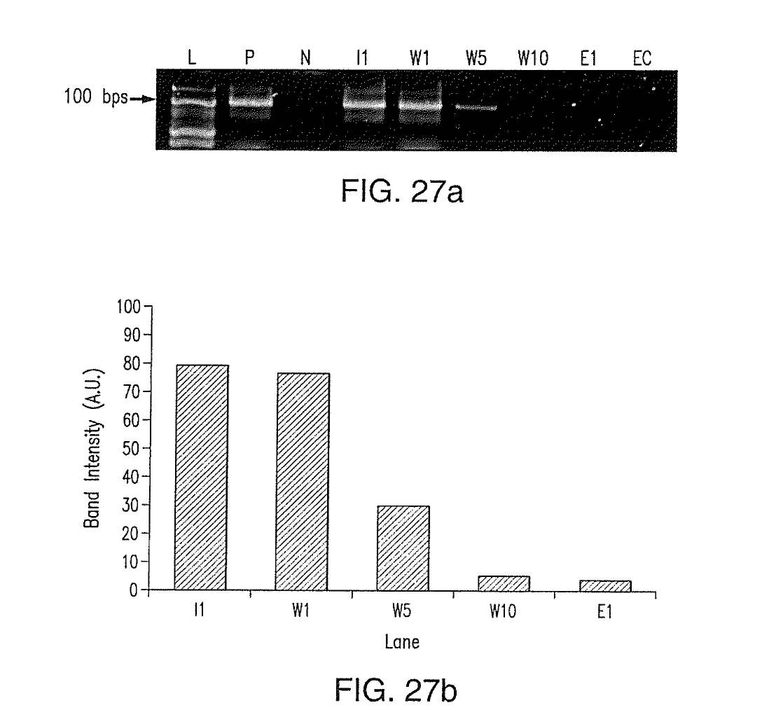

To verify that the isolated ssDNA strands were specifically bound to IgE, the example was repeated using fresh NHS beads with no protein coupled to the surfaces. The gel image (FIG. 27a) and bar graph (FIG. 27b) show bright bands during incubation and earlier washing steps (lanes I1-W5) and no band during later washing and elution steps (lanes W10-E1). This indicates that ssDNA were very weakly bound on the NHS bead surfaces and removed by stringent washings. Hence, the 97-mer ssDNA collected in the previous example shown in FIG. 26 are mostly likely IgE-binding ssDNA isolated from the random mixture. Similar to the previous result, no band seen in lane EC indicates that the gel in the channel prevented the undesired ssDNA from entering the enrichment chamber.

1×PBS and 0.5×TBE buffers were tested as exemplary electrolytes for the electrophoretic transport of DNA through the gel-filled microchannel. PBS buffer is a strong electrolyte (electrical conductivity: 15 mS/cm), and as it is commonly used for other steps in the process its use in electrophoresis can simplify the enrichment process. Alternatively, TBE buffer (approximate electrical conductivity: 350 μS/cm) can be used (as it is an electrolyte commonly used in gel electrophoresis applications).

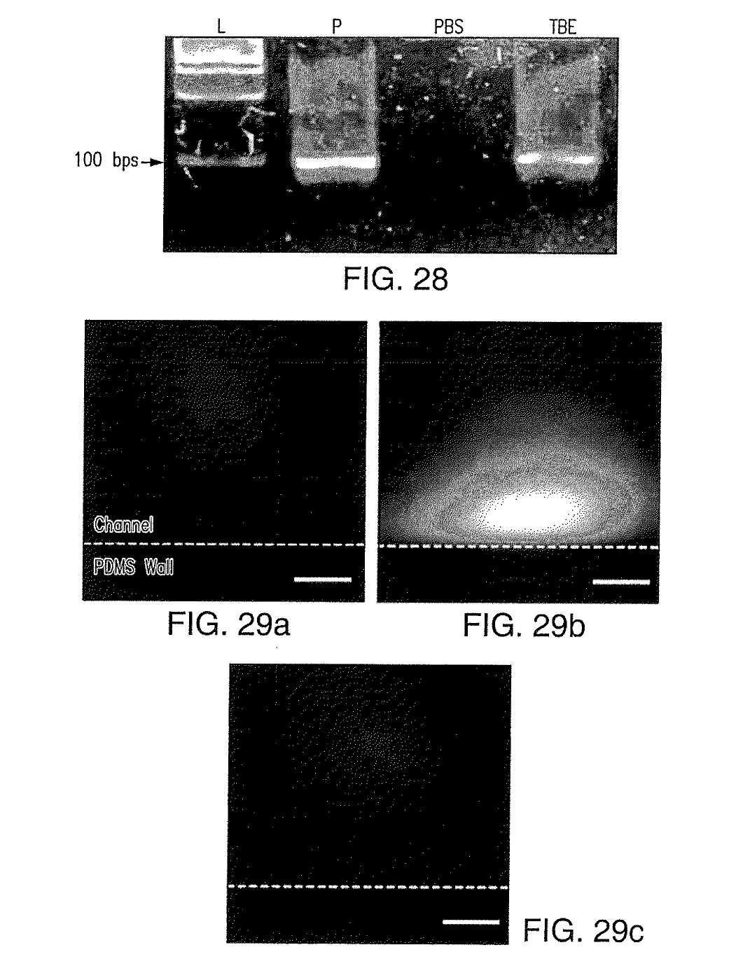

As shown in FIG. 28, when PBS was used as the electrophoresis buffer, no band was visible in corresponding lane, indicating that DNA strands were not transported to the enrichment chamber. However, when using TBE buffer, DNA strands migrated to the enrichment chamber effectively, as indicated by a distinctly visible band seen in lane TBE. This can be explained by noting that although PBS has a much higher conductivity than TBE, the salt ions (i.e., Na⁺ and Mg²⁺) present in PBS buffer shield DNA strands and neutralize their negative charges, preventing them from migrating toward the anode (i.e., enrichment chamber). As a result, 0.5×TBE buffer was used for electrophoretic transport of DNA in this microchip.

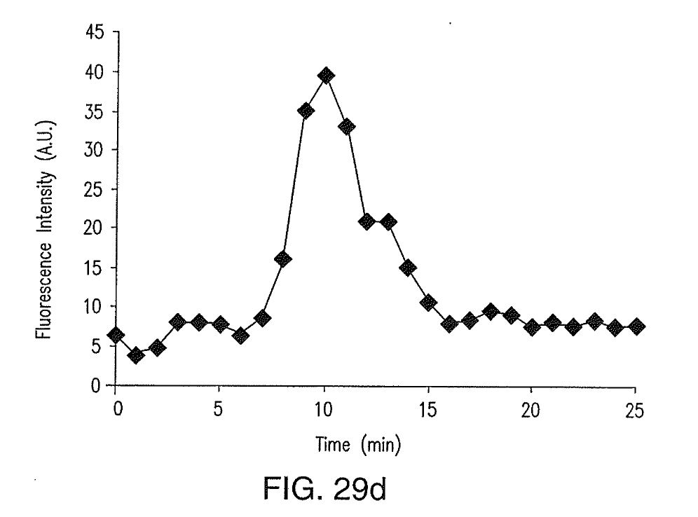

Time required to electrophoretically transport ssDNA from the isolation to enrichment chambers was determined. The fluorescence micrographs obtained during electrophoretic transport of fluorescently labeled ssDNA strands at different times monitored at the center of the gel-filled channel are shown in FIGS. 29a-29c. The peak in the fluorescence intensity profile at 10 minutes indicates that the ssDNA were migrating at a speed of approximately 1 mm/min through the gel-filled channel (FIG. 29d). As the distance between the two chambers is approximately 20 mm, at least 20 minutes was required to electrophoretically transport the ssDNA to the enrichment chamber for this microchip. To assess the efficiency of the example setup, the electrophoretic mobility of the DNA was calculated using the measured DNA velocity according to the equation V=μE, where μ is the electrophoretic mobility, V is the velocity of migrating DNA, and E is the applied electric field (i.e., 25 V/cm in such example). The electrophoretic mobility of ssDNA was thus calculated to be 6.67×10⁻⁵ cm²/Vs, which is within the range of reported values in the literature.

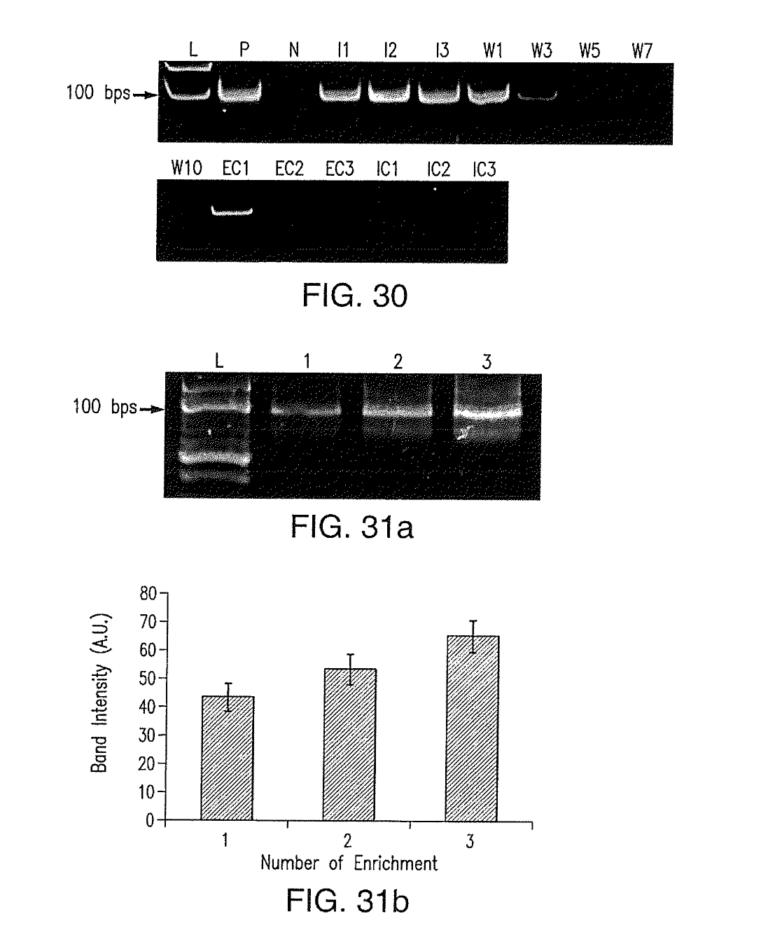

An entire round of isolation and enrichment of IgE-binding ssDNA in a single microchip was performed. Random ssDNA library was exposed to IgE-coated beads, weakly bound strands were washed away, and aptamer candidates were thermally eluted and electrophoretically transported to the enrichment chamber. To analyze the results, eluent was collected from each step (i.e., incubation: I, washing: W, elution: E) as well as the buffer used to wash the two chambers (i.e., isolation chamber: IC, elution chamber: EC) after the processes were completed. These eluents were then chemically amplified using PCR and visualized using slab gel electrophoresis. The electropherogram visualizing the amplified eluents are shown in FIG. 30. DNA that did not bind to IgE during the incubation process is indicated by the bands in lanes I1-I3. As expected, these bands display high levels of fluorescent intensity, as most of the random DNA did not bind to IgE. The decrease in band intensity from lane W1 to W10 indicates that ssDNA strands having low binding affinity to IgE were gradually removed as the beads were continuously washed with buffer. The bright band in lane EC 1 and dimmer band in lane IC1 indicate that the majority of the thermally eluted ssDNA having high binding affinities to IgE were electrophoretically transported to the enrichment chamber.

To investigate the ability of the microchip to enrich IgE-binding DNA, multiple rounds of ssDNA enrichment were performed on the chip. The isolated IgE-binding ssDNA strands were repetitively enriched via electrophoretic transport. A gel electropherogram of amplified eluents collected after 1 round (lane 1), 2 rounds (lane 2), and 3 rounds (lane 3) of enrichment in the microchip is shown in FIG. 31a. With the increasing the number of enrichment rounds a higher concentration of DNA was detected in the enrichment chamber (FIG. 31b). In addition, no damage to the gel-filled microchannel was observed after the multiple rounds of enrichment.

### Example 4

This Example demonstrates the use of a microdevice having a similar structure as that in Example 3, but using chemical elution rather than thermal elution to release the bound candidate aptamers.

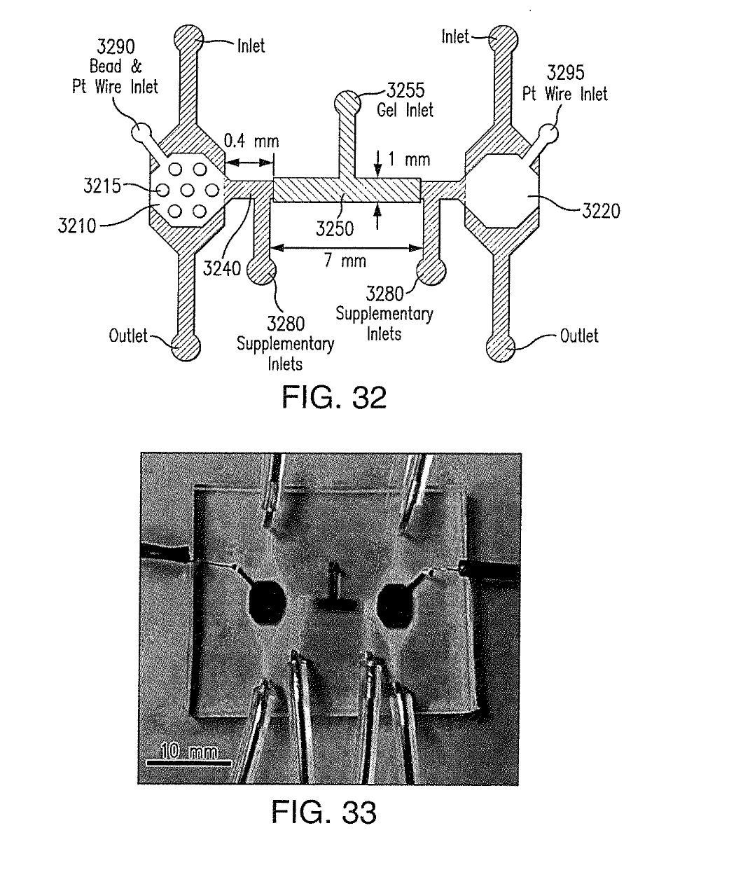

The microdevice used in this Example includes a selection/isolation chamber 3210 and an enrichment chamber 3220 each having a depth of 200 μm and a volume of 5 μL, the chambers being connected by a microchannel 3240 (1 mm×7.8 mm×40 μm) (as shown in FIG. 32). Microbeads 3215 (diameter: 100 μm) are retained by a dam-like structure (weir, height: 40 μm) in the isolation chamber. The channel 3240 is partially filled with a gel 3250 (3% agarose or 12% polyacrylamide) through a gel inlet 3255. An additional length of channel (0.4 mm) thermally insulates the gel from the heated chambers when heating is used to elute the ssDNA from the beads. Supplementary inlets 3280 are used to fill these areas with buffer. Platinum (Pt)-wire electrodes are inserted through Pt inlets (3290 and 3295) to provide a potential difference across the chambers for electrophoresis. The microdevice can be fabricated by a procedure substantially similar to that described in Example 3 (in connection with FIG. 23), except that the sensor and heater were not introduced. An image of the fabricated microdevice is shown in FIG. 33.

In the example, human myeloma IgE (Athens Research & Technology) was dissolved in 1× phosphate-buffered saline (PBS buffer) to a final concentration of 1 μM. NHS-activated microbeads were purchased from GE Healthcare and functionalized with IgE. An 87-mer ssDNA library having random sequences (5′-GCC TGT TGT GAG CCT CCT GTC GAA-N40-TTG AGC GTT TAT TCT TGT CTC CC-3′) (SEQ ID NO:11), and fluorescently labeled IgE-specific ssDNA aptamer D17.4 primers were purchased from Integrated DNA Technologies. The mixtures of ssDNA were prepared by mixing 1 μL of the random library (100 μM) and 1 μL of the aptamer D17.4 (0.1 μM) in 98 μL of PBS buffer.

Isolation and enrichment of desired ssDNA molecules in a randomized ssDNA mixture was carried out as follows. The IgE-beads were loaded in the isolation chamber using a syringe through a bead inlet to fill approximately 30% of the chamber volume. After loading, the beads were washed for 5 minutes with PBS buffer at a flow rate of 20 μL/min using a syringe pump. Throughout the examples, a DNA mixture having 0.1% of IgE-specific aptamer D17.4 was used. The ssDNA mixture was introduced to the isolation chamber and collected from the outlet of the isolation chamber in 3 separate samples (sample volume: ˜33 μL). PBS buffer was introduced to the chamber at 20 μL/min to release weakly bound DNA strands from the IgE-beads, and the waste solution is collected in 10 separate samples at the outlet (sample volume: ˜30 μL). To elute strongly bound DNA strands from the beads, 5 μL of 0.1 M NaOH in 0.5×TBE buffer was introduced into the chamber and incubated for 5 minutes. Platinum electrodes were inserted into the Pt inlets in each chamber and an electric field of 25 V/cm was applied. The strands were then electrophoretically transported to the enrichment chamber through the gel-filled channel. Following electrophoretic DNA transport, the isolation chamber was washed with PBS buffer to remove undesired DNA molecules. To concentrate the IgE-binding ssDNA in the enrichment chamber, the isolation and electrophoretic transport processes was repeated. Samples of eluent containing ssDNA molecules were collected throughout the example and amplified using polymerase chain reaction (PCR) off-chip. Slab-gel electrophoresis was used to evaluate the concentration of DNA eluted throughout the process by comparing the band intensities for each sample. A fluorescence microscope was used to monitor the electrophoretic transport of ssDNA through the gel-filled channel.

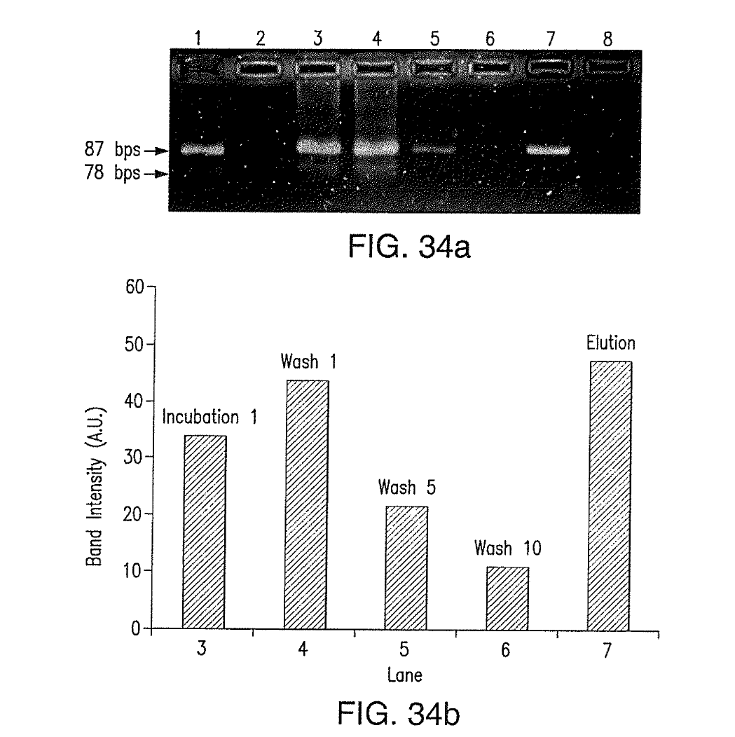

IgE-binding ssDNA was first isolated from the randomized ssDNA mixture in the isolation chamber. FIG. 34a shows a gel electropherogram of the PCR products of eluents collected during the isolation. In the image, 87-bp bands represent the random ssDNA in the pool while 78-bp bands represent D17.4 aptamer. Lanes 1 and 2 are positive (a mixture of random ssDNA and D17.4 aptamer) and negative (whole PCR mixture except DNA template) controls, respectively. The two distinct bands seen in lane 1 are for a PCR product of a mixture of random ssDNA containing D17.4 aptamer. No bands are seen in lane 2, indicating that reagents used were not contaminated by undesired DNA molecules.

The band intensity profile of the 87-mer ssDNA during incubation, consecutive washes, and elution is shown in FIG. 34b. 87-mer ssDNA that did not bind to the IgE-beads during the incubation step are indicated by the bright band in lane 3. The decreasing intensity of the bands from lane 4 to lane 6 indicates that as washing continued, loosely bound 87-mer ssDNA molecules were removed from the bead surfaces, increasing the stringency of the isolation. Strongly bound 87-mer ssDNA molecules were eluted from the IgE-bead surfaces by 0.1 M NaOH as shown in lane 7.

The enrichment chamber was washed with buffer just prior to electrophoretic migration of isolated DNA. To determine if the gel-filled channel prevents contamination of the enrichment chamber with unwanted DNA from the isolation chamber, the buffer was PCR amplified and analyzed using gel electrophoresis (FIG. 34a, lane 8). No band is shown in that lane, indicating the gel effectively blocked the unwanted ssDNA from contaminating the enrichment chamber.

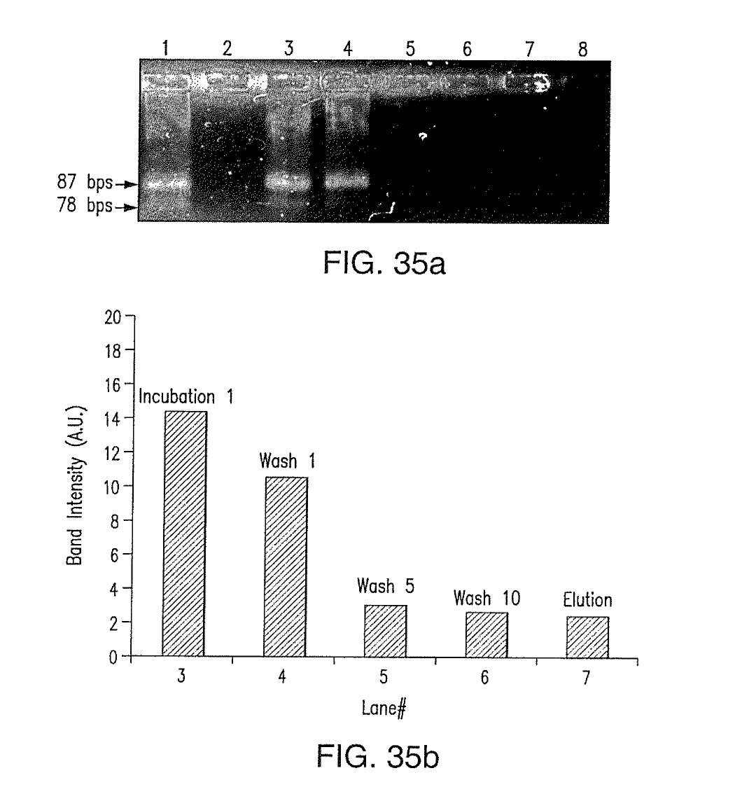

To verify that the eluted 87-mer ssDNA is IgE-specific, the isolation was repeated using fresh NHS beads without functionalizing the surfaces. Following all buffer washes, samples of buffer were PCR amplified using the same conditions as with the previous test (FIG. 35). The gel image and band profile show bright bands for incubation (lane 3) and wash 1 (lane 4) while no bands are observed in washes 5 and 10 (lanes 5 and 6), and elution (lane 7). This indicates that ssDNA were bound on the NHS-beads very weakly and released during earlier washing steps. Hence, the 87-mer ssDNA collected in the previous example shown in FIG. 34 are IgE-binding ssDNA isolated from the random mixture.

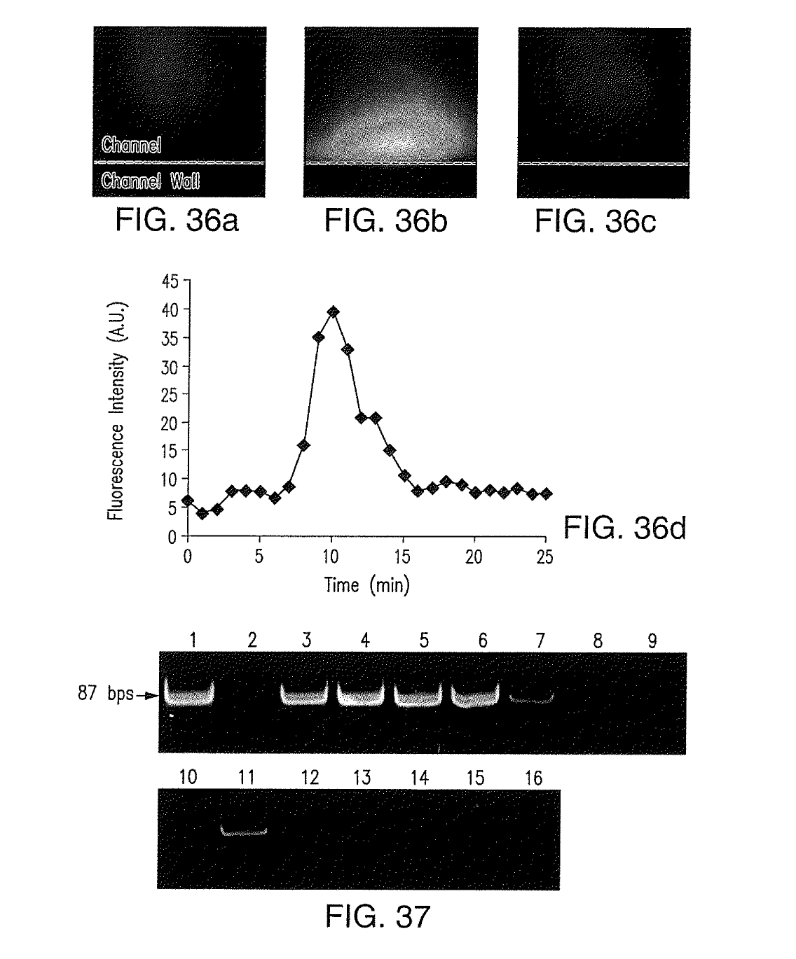

The electrophoretic transport of isolated ssDNA through the gel-filled microchannel was then investigated. Two Pt electrodes connected to a DC power supply were inserted in the Pt inlets of the isolation and enrichment chambers to form a cathode and an anode, respectively. The electrophoretic transport of the ssDNA through the gel-filled channel was monitored using an inverted epifluorescence microscope. The fluorescence micrographs obtained at different times at the center in the channel are shown in FIGS. 36a-36c. The peak in the fluorescence intensity profile at 10 minutes indicates that the eluted ssDNA were migrating at a speed of approximately 1 mm/min through the gel-filled channel (FIG. 36d). As the distance between the two chambers is 20 mm, it took approximately 20 minutes to transport the ssDNA to the enrichment chamber.

Following the electrophoretic transport of IgE-binding ssDNA for 20 minutes, the two chambers were thoroughly washed with PBS buffer and the waste solutions were collected. A gel electropherogram of the collected eluents is shown in FIG. 37. Bands in lanes 3, 4, and 5 correspond to ssDNA that did not bind to the IgE-beads during incubation. The band intensities in lanes 6-10 decrease due to continuous washing. The bright band in lane 11 corresponds for the electrophoretically transported ssDNA to the enrichment chamber. On the other hand, a very weak band is shown in lane 14 representing waste collected from the isolation chamber after electrophoresis. This indicates that the majority of the captured ssDNA have migrated from the isolation chamber to the enrichment chamber.

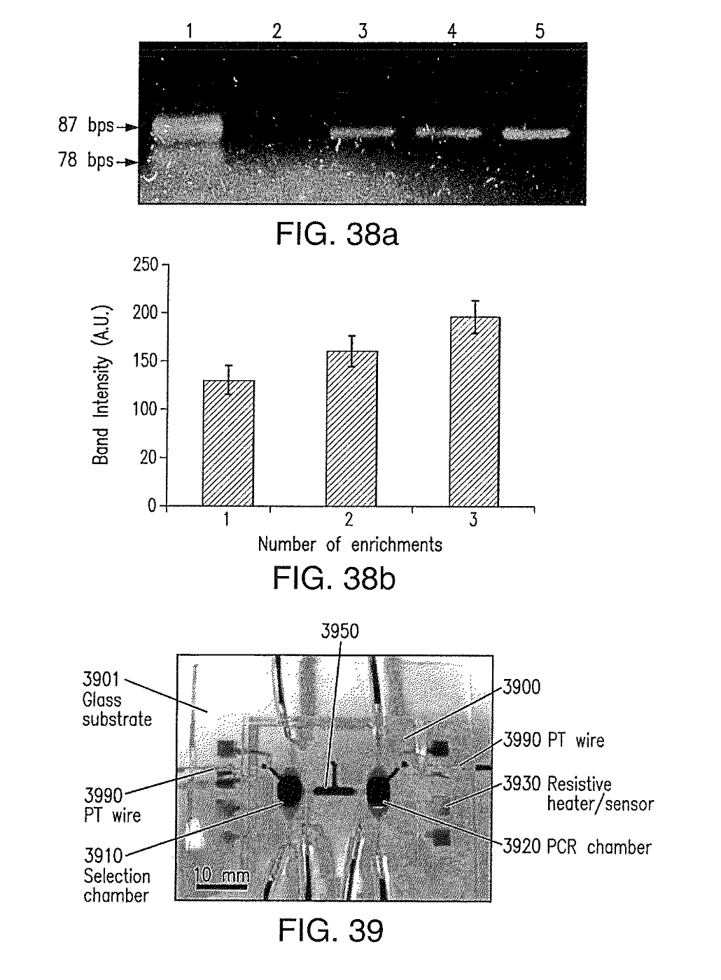

To investigate the ability of the chip to enrich DNA, multiple rounds of ssDNA isolation and transport were performed on a single chip. Following a round of isolation and transport, fresh IgE-beads were introduced to the isolation chamber. The captured ssDNA was then concentrated in the enrichment chamber by repetitive isolation and transport. A gel electropherogram for eluents collected after 1 round (lane 3), 2 rounds (lane 4), and 3 rounds (lane 5) of enrichment in different chips is shown in FIG. 38a. The band intensity profile shows an increase in band intensities as the number of enrichment rounds increases, indicating a higher concentration of DNA following a greater number of enrichments (FIG. 38b).

### Example 5

In this Example, gel-based electrophoretic nucleic acid transport, bead-based nucleic acid isolation, and polymerase chain reaction (PCR) are combined to simplify microchip design, fabrication, and operation by eliminating the need for complex flow handling components. The process includes the selection and electrophoretic transport as described above in connection with FIG. 3, and further includes amplifying the electrophoretically transported candidate aptamers in the enrichment chamber using reverse-primer coated microbeads.

FIG. 39 shows an image of the microchip used in this Example. The microchip 3900 used in this test include a selection chamber 3910 and enrichment/amplification chamber 3920 each having a volume of 5 μL and weir structures of 40 μL in depth for trapping beads of diameter about 100 μm. Integrated resistive heaters and sensors 3930 (Cr/Au:5/100 nm) were used to control the temperature in the chambers during thermal elution in the selection chamber and thermal cycling in PCR. The two chambers were connected by an agarose-filled channel 3950 (7 mm×0.8 mm×40 μm). An electric field (25 V/cm) for DNA electrophoresis is generated by platinum electrodes 3990. The microchip was prepared using microfabrication techniques, as illustrated in Examples 3 and 4.

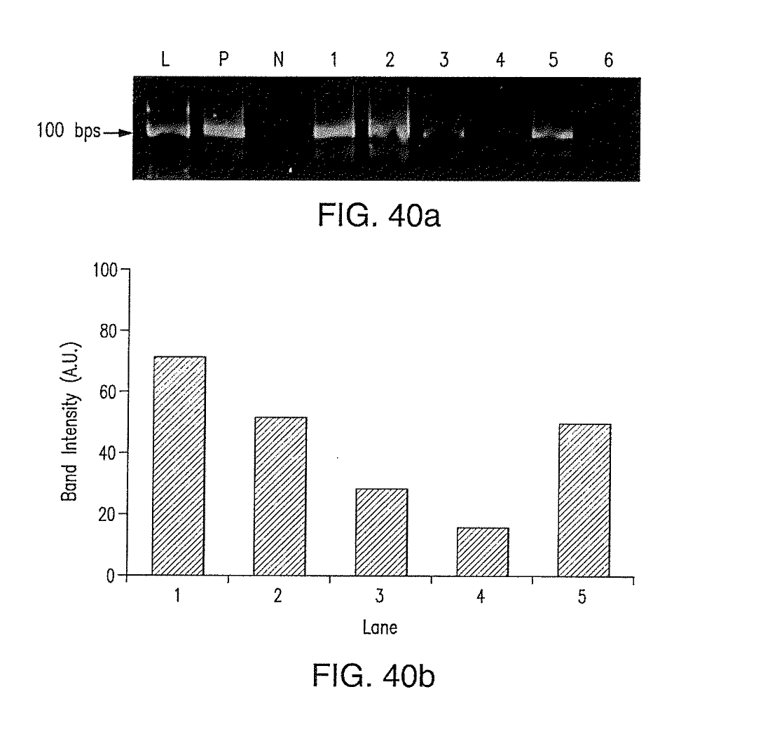

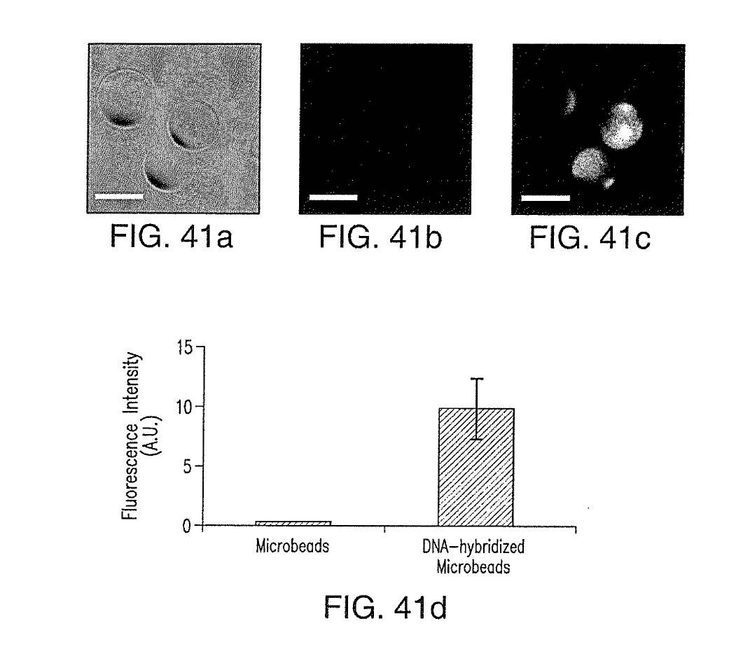

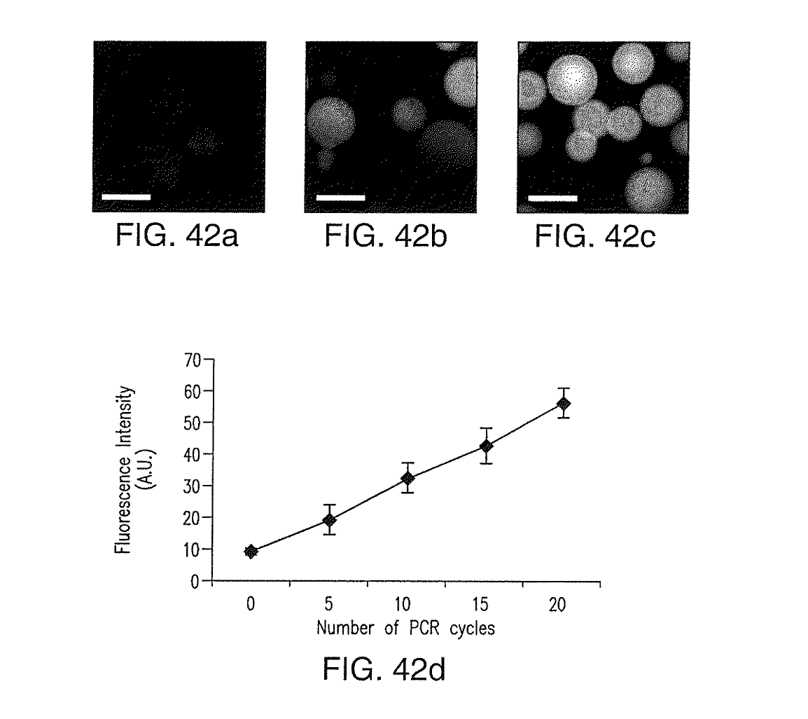

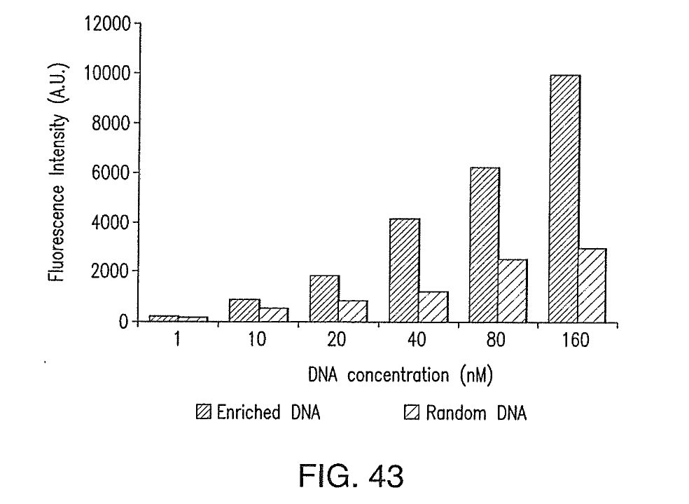

Amplified eluents from each step were visualized using gel electrophoresis and intercalating dyes, as shown in FIGS. 40a and 40b. DNA that did not bind to the beads is visualized in lane 1, and no fluorescence in lane 6 indicates no DNA contamination of the amplification chamber from selection. The decrease in band intensity from lanes 2 to 4 indicates that weakly bound ssDNA were gradually removed from the beads during washing, while the band in lane 5 represents ssDNA that was strongly bound to IgE. An electric field applied across the two chambers electrophoretically transported fluorophore-labeled ssDNA to the PCR chamber. There, reverse-primer coated microbeads captured the DNA as shown in FIG. 41. FIG. 41a shows micrograph of beads in the PCR chamber. Fluorescence images of the beads before and after hybridization of fluorophore-labeled ssDNA are shown in FIGS. 41b and 41c, respectively. The scale bars in FIGS. 41a-41c represent 100 μm. FIG. 41d is a bar graph depicting the fluorescence intensities of the beads before and after ssDNA hybridization. The DNA was amplified to generate a fluorescent signal proportional to DNA concentration as shown in FIG. 42 (FIGS. 42a-42c show fluorescence intensity of beads after 0, 10, 20 PCR cycles, respectively (scale bar in 42(a)-(c): 100 μm); FIG. 42d is a bar graph showing fluorescence intensities of the beads after given number of PCR cycles.) The increased fluorescence intensity of beads incubated with the enriched ssDNA indicates the selected DNA bind to IgE considerably more strongly than the random library, as shown in FIG. 43, which compares the binding affinity between enriched DNA and random DNA to IgE-coated beads at same concentration.

In further tests, the amplified strands were separated (e.g., by eluting) from the beads in the PCR chamber and electrophoretically transported back into the isolation chamber, such that additional selection-amplification rounds can be performed. During multiple rounds of the aptamer selection-transport-amplification process, no damage was observed to the agarose gel in the channel indicating that the gel physically separated the two chambers while eliminating cross-contamination of buffers by selectively transporting nucleic acids via electrophoresis. The binding affinity of the randomized nucleic acid pool to IgE significantly improved to 13 nM after 4 rounds of aptamer selection. An enriched mixture of 87-mer and 78-mer nucleic acids was used for the binding affinity measurements. Multiple rounds of isolation and enrichment of aptamers against steroid and MCF-7 cells on the microchip were also tested. Results showed this on-chip approach can be simple yet versatile enough to select aptamers against a variety of functional molecules.

### Example 6

This Example illustrates a MEMS-based SNP genotyping method that performs PCR, SBE, and desalting reactions on microbeads in a single microchamber, as previously described in connection with FIG. 4.

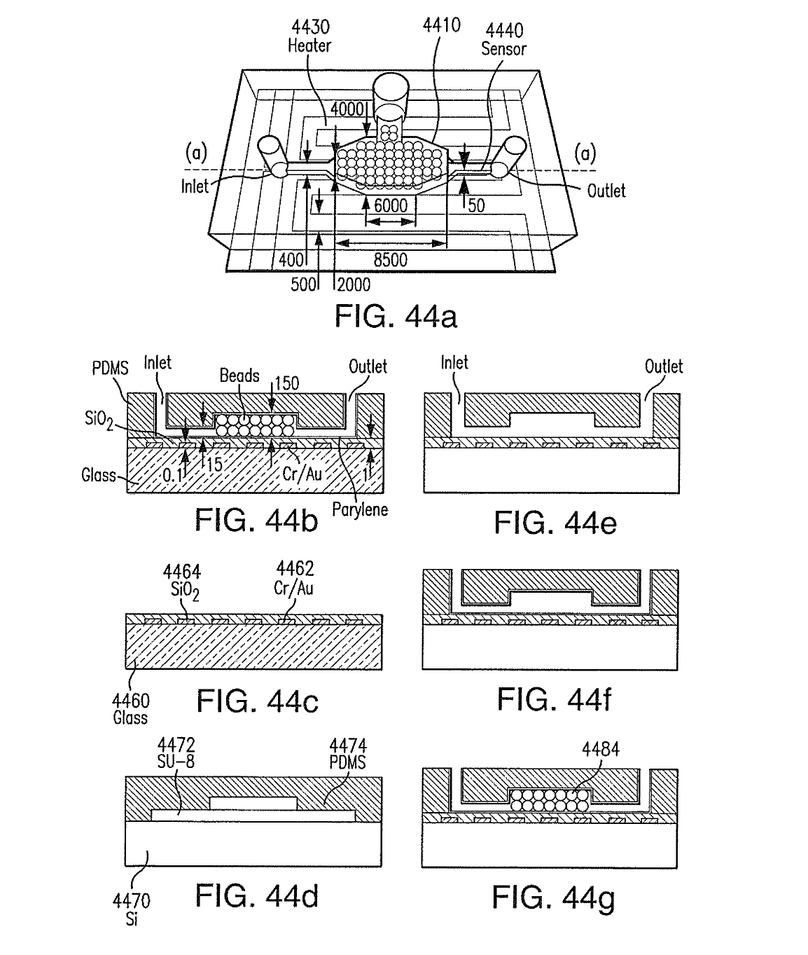

The microfluidic device used in this Example includes a microchamber 4410 formed by PDMS situated on a microheater 4430 and temperature sensor 4440 (FIGS. 44a and 44b). The microchamber (FIGS. 44a and 44b, 150 μm in height) with an approximately 5 μL volume contains weirs (FIGS. 44a and 44b, 15 μm in height) to retain microbeads (50-80 μm in diameter) during wash steps. The surfaces of microchamber are coated with Parylene C to prevent evaporative loss of reactants. A resistive sensor (16.5 mm L×50 μm W) is located beneath the center of the chamber, and a resistive serpentine-shaped heater (296 mm L×500 μm W) surrounds the temperature sensor. Thus, the chamber is heated with its temperature near the center measured by the sensor to complete a closed-loop temperature control setup.

The temperature control part of the microchip was fabricated using standard microfabrication techniques. Briefly, a glass slide 4460 (Fisher HealthCare, Houston, Tex.) was cleaned by piranha. Chrome (10 nm) and gold (100 nm) thin films 4462 were deposited by thermal evaporation and patterned by wet etching. Then, a passivation layer of 1 μm of silicon dioxide 4464 was deposited using plasma-enhanced chemical vapor deposition (PEC VD). Finally, contact pads for wire bonding, connecting the instruments to the on-chip sensor and heater, were opened by etching the oxide layer using hydrofluoric acid (FIG. 44c).

Separately, the microfluidic chamber was fabricated from PDMS (Sylgard 184, Dow Corning Inc. Midland, Mich.) using soft lithography techniques. SU-8 photoresist 4472 (MicroChem. Corp., Newton, Mass.) was spin-coated and patterned on a silicon wafer 4470 to form mold-defining microfluidic features. Next, a PDMS prepolymer solution (base and curing agent mixed in a 10:1 ratio) was cast onto the mold and cured on a hotplate at 72° C. for 1 hour (FIG. 44d) to form the wall of the microchamber 4474.

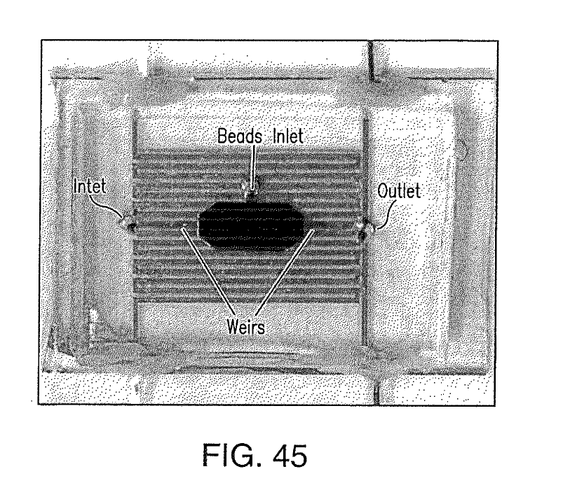

Subsequently, the inlet and outlet were punched on the resulting sheet bearing the microfluidic features, which was then bonded to the temperature control chip after treatment of the bonding interfaces with oxygen plasma for 15 seconds (FIG. 44e). Finally, the surface of the microchamber was coated with a thin layer of Parylene C via chemical vapor deposition (FIG. 44f), prior to packing streptavidin beads 4484 (FIG. 44g). An image of a fabricated device is shown in FIG. 45.

All chemicals used in this Example were purchased from Sigma-Aldrich (St. Louis, Mo.) unless otherwise indicated. Streptavidin beads (Pierce Streptavidin Plus UltraLink Resin) were obtained from Thermo Fisher Scientific Inc. (Rockford, Ill.). Dideoxynucleotide triphosphates (ddNTPs) were purchased from Jena Bioscience GmbH (Jena, Germany). Deoxynucleotide triphosphates (dNTPs) and GoTaq Flexi DNA Polymerase were obtained from Promega Corp. (Madison, Wis.). Thermo Sequenase was purchased from GE Healthcare (Piscataway, N.J.). Template DNA, including a mutated type (5′-CCT CAC CAC CAA CTT CAT CCA COT TCA CCT TGC CCC ACA GGG CAG TA [symbol] A CAG GAG TCA GAT GCA CCA TOG TOT CTG TTT GAG GTT GCT AGT GAA CAC AGT TGT GTC AGA AGC AAA TGT AAG CAA TAG ATG GCT CTG CCC TGA CT-3′ (SEQ ID NO:12), the SNP site is underlined and SBE primer annealing site is italic) and an unmutated type (5′-CCT CAC CAC CAA CTT CAT CCA CGT TCA CCT TGC CCC ACA GGG CAG TA [symbol] [symbol] T CAG GAG TCA GAT GCA CCA TGG TGT CTG TTT GAG GTT GCT AGT GAA CAC AGT TGT GTC AGA AGC AAA TGT AAG CAA TAG ATG GCT CTG CCC TGA CT-3′ (SEQ ID NO:13), the SNP site is underlined and SBE primer annealing site is italic) of the HBB gene, double biotin modified reverse primer (5′-double biotin-AGT CAG GGC AGA GCC ATC TA-3′) (SEQ ID NO:14), fluorescein (FAM) modified forward primer (5′-FAM-CCT CAC CAC CAA CTT CAT CC-3′, M.W.=6651) (SEQ ID NO:15), and SBE primer (5′-ACG GCA GAC TTC TCC-3′, M.W.=4513) (SEQ ID NO:16) were synthesized and purified by Integrated DNA Technologies (Coralville, Iowa).

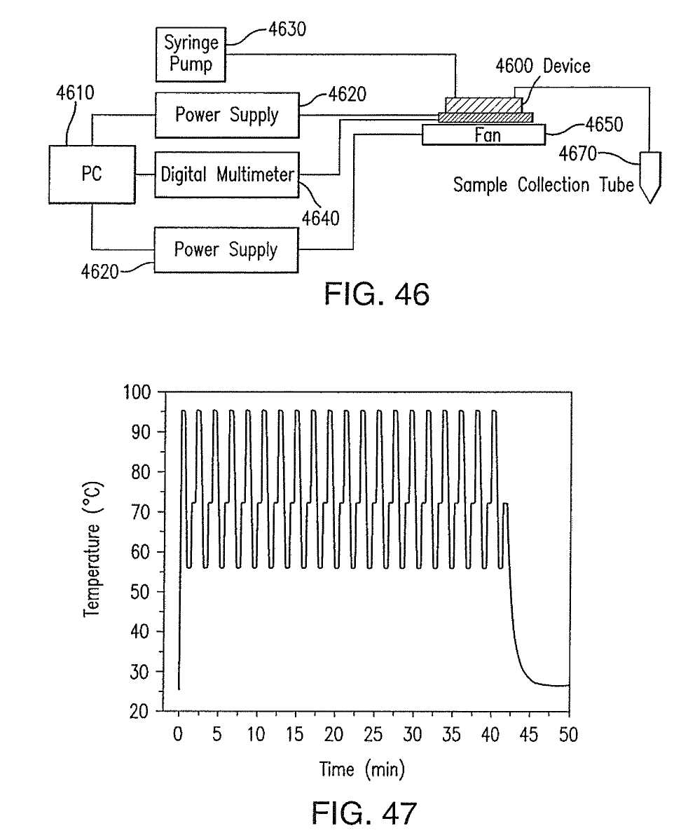

Closed-loop temperature control of the microchamber was achieved using the integrated temperature sensor, heater, and a fan 4650 under the microchip 4600 with a proportional-integral-derivative (PID) algorithm implemented in a LabVIEW (National Instruments Corp., TX) program on a personal computer 4610. The resistance of the sensor was measured by a digital multimeter 4640 (34420A, Agilent Technologies Inc., CA), and the heater and fan were connected to two DC power supplies 4620 (E3631, Agilent Technologies Inc., CA) respectively. The inlet was connected to a syringe that contained reaction buffer or washing buffer driven by a syringe pump 4630 (KD210P, KD Scientific Inc., MA). The outlet was connected to a microcentrifuge tube 4670 for collection of genotyping product to MALDI-TOF MS or waste. All fluorescent images of beads were taken using an inverted epifluorescence microscope (Diaphot 300, Nikon Instruments Inc., NY) with a CCD camera (Model 190CU, Micrometrics, NH), after removing the device from the fan (FIG. 46).

The streptavidin beads in the microchamber were rinsed with binding and washing (B&W) buffer (5 mM Tris-HCl, 0.5 mM EDTA, 1 M NaCl, and 0.01% Tween 20, pH=7.5). The reverse primer (50 pmol) in B&W buffer was introduced and incubated with the beads for 30 min, followed by washing with B&W buffer at 10 μL/ruin for 10 min.

Bead-based PCR was performed for 30 thermal cycles as follows: 95° C. for 15 s, 56° C. for 30 s, and 72° C. for 30 s. A 5 μL sample of PCR reactants was introduced twice, prior to cycling and between 15th and 16th cycle, and each sample consisted of 0.08 pmol of template, 8.33 pmol of forward primer, 1× GoTaq Flexi Buffer, 0.83 units of GoTaq Flexi DNA Polymerase, 1.67 nmol of dNTP and 6.25 nmol of MgCl₂ (1.25 mM). The microbeads were then rinsed with 0.15 mM NaOII in B&W buffer at 5 μL/min for 10 min to elute template ssDNA, followed by a rinse of pure B&W buffer at 10 μL/min for 10 min, leaving complementary ssDNA on the beads.

To perform SBE, the SBE primer targeting the SNP on the complementary sequence of exon 1 of the HBB gene was extended by a single base in the microchamber using ddNTPs. A 5 μL sample of SBE reactants was introduced to the microchamber twice, prior to SBE and between 5th and 6th thermal cycle, and underwent 10 thermal cycles as follows: 90° C. for 15 s, 40° C. for 30 s, and 70° C. for 30 s. Each SBE reactant consisted of 6.67 pmol of primer, 16.67 pmol of ddNTP, 1× Thermo Sequenase reaction buffer and 2.67 units of Thermo Sequenase. The microchamber was then rinsed using B&W buffer at 5 μL/min for 10 min, followed by desalting with DI water at 5 μL/min for 20 min. Finally, the microchamber was incubated at 95° C. for 1 min, followed by a rinse with DI water at 20 μL/min and 95° C. for 3 min, to elute the hybridized primer.

The temperature-resistance relationship of the thin-film gold temperature sensor was calibrated following fabrication. The calibration data showed that the measured resistance (R) of the sensor exhibited a highly linear relationship with temperature (T), which can be fitted to R=R₀[1+α(T−T₀)], where R₀ is the sensor resistance at reference temperature T₀, and α is the temperature coefficient of resistance (TCR) of the sensor. The TCR was determined to be 3.06×10⁻³° C.⁻¹ for a typical chip, which had a reference resistance of 83.44Ω at a reference temperature of 21.9° C. Time-resolved tracking of on-chip thermal cycling showed that the chamber temperatures attained specified setpoints via control of the on-chip heater and off-chip fan quickly and precisely (FIG. 47). The thermal time constant of a typical temperature control chip was 126 s based on an exponential fit. The time constants of closed loop temperature control (based on an exponential fit) were 1.4 s for heating from 56° C. to 72° C., 1.9 s for heating from 72° C. to 95° C. and 8.7 s for cooling from 95° C. to 56° C., which represented a significant improvement over typical time responses of conventional PCR thermal cyclers (e.g., 6 s for heating from 56° C. to 72° C., 8 s for heating from 72° C. to 95° C., and 16 s for cooling from 95° C. to 56° C. for the Eppendorf Mastercycler® Personal used in related examples below).

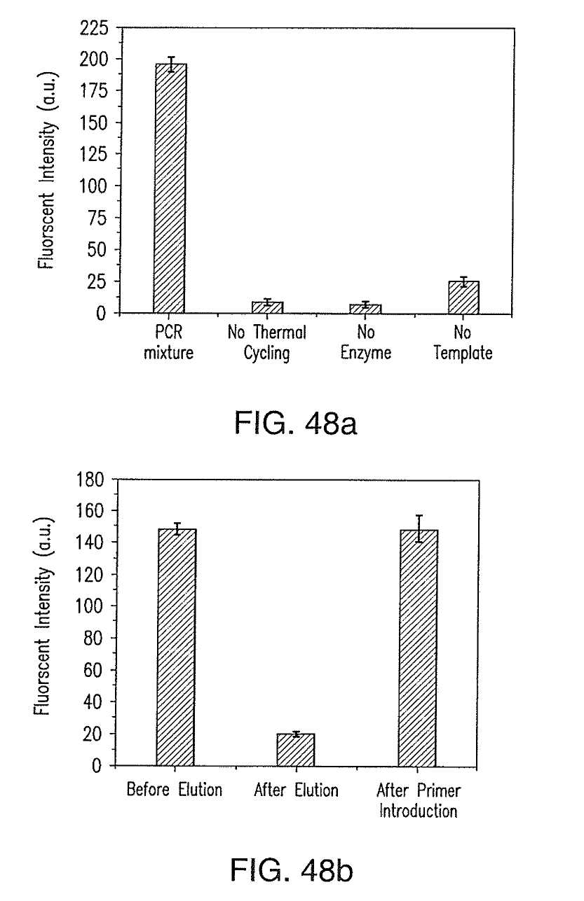

To characterize bead-based PCR, reactants were thermally cycled on-chip and fluorescent bead intensity was then measured and compared to control tests. To obtain consistent results under controlled conditions, template DNA was used as the target sequence for the characterization. After B&W buffer washing, the fluorescent intensity of beads was significantly higher than those without thermal cycling, enzyme or templates, which were only 5%, 7% and 16% of the original test (FIG. 48a). This indicates that the bead-based PCR process did amplify template DNA and that the fluorescently modified primer enables monitoring of this step of the SNP genotyping procedure.

Prior to SBE, template ssDNA generated during PCR were removed from the bead-bound complementary DNA. To test the efficiency of the chemical elution method, the template ssDNA was first amplified using fluorescently labeled forward primers and double biotinylated reverse primers in a conventional thermal cycler, and the amplification product was immobilized onto the streptavidin beads, which were packed in the microchamber afterwards. The fluorescent intensity of the beads was then measured before and after rinsing with buffer. The fluorescent intensity of rinsed beads was 87% lower than that of pre-elution beads (FIG. 48b), indicating that most template ssDNA had been removed from the bead surface. To further demonstrate that the template ssDNA had been removed from the beads, rather than the dsDNA, 5 μL of 5 μM FAM-modified forward primers in 1×PCR buffer was introduced into the microchamber. After incubating at 56° C. for 1 min, followed by washing with B&W buffer, the fluorescent intensity of the beads was similar to that before introduction of the NaOH elution (FIG. 48b), which suggests that complementary ssDNA remained bound to the beads following the elution of template ssDNA. These results indicate a sufficiently high on-chip chemical elution efficiency using NaOH.

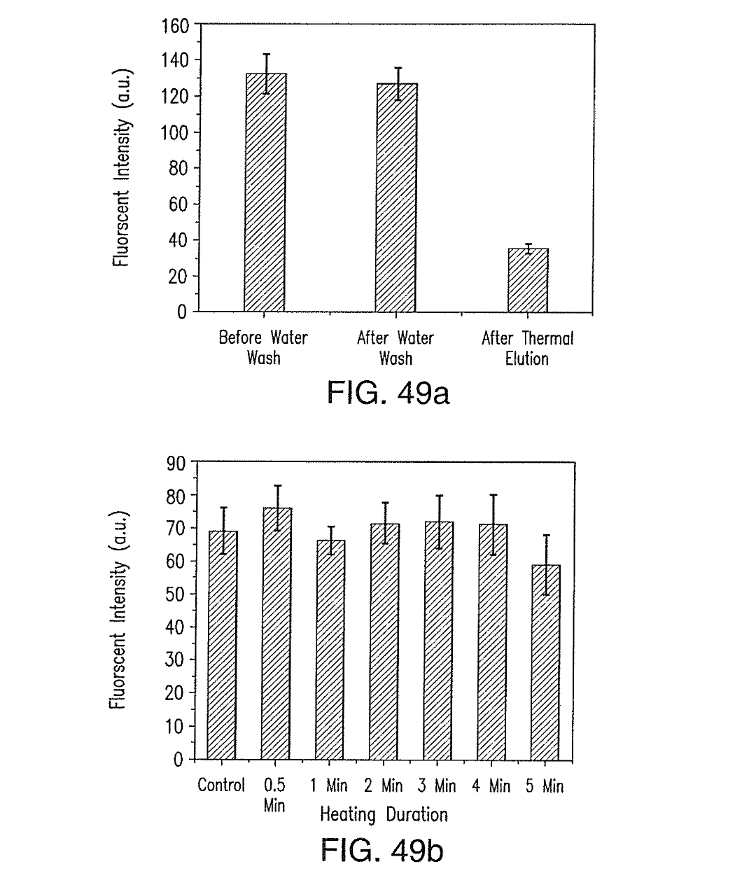

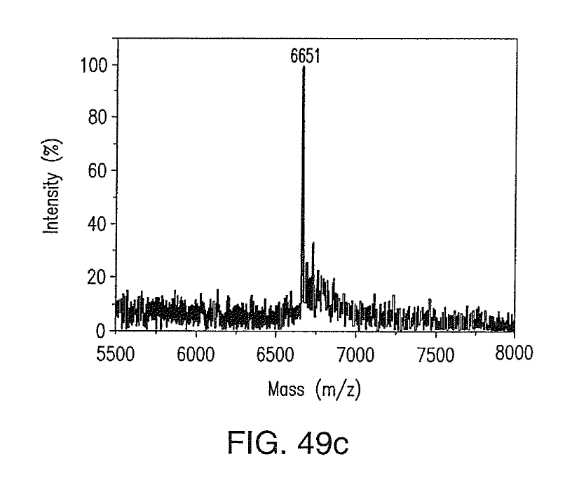

To generate a DNA solution prior to detection with MALDI-TOF MS, hybridized primers were desalted and then thermally eluted into DI water. The effect of desalting and the efficiency of the thermal elution method were tested to ensure that DNA loss during this step would not compromise detection by MS. The fluorescently labeled forward primer in B&W buffer was first hybridized to the ssDNA on the beads and desalted with DI water. The fluorescent intensity of the beads was then measured before and after rinsing at 95° C., and the elution product was manually pipetted to a MALDI plate and tested using MALDI-TOF MS. During desalting, the microchamber was rinsed with DI water, and the fluorescent intensity remained at 95.5% of pre-desalting intensity (FIG. 49a). The chamber temperature was then elevated to elute hybridized primers. After elution, the fluorescent intensity of the beads was only 26% of pre-desalting intensity and 28% of pre-elution intensity (FIG. 49a). To control for effects of temperature on fluorescent intensity, fluorescently labeled microbeads were heated in the thermal cycler for different durations. As shown in FIG. 49b, heating for 4 min did not generate a noticeable change in fluorescent intensity, which showed that the intensity of the fluorescent label was stable in response to elevated temperatures, and that elution of primers was indeed the reason for the decrease in fluorescent intensity. Furthermore, following MALDI-TOF MS, a distinct mass spectral peak at 6651 m/z (FIG. 49c) indicated effective desalting efficiency. The results of the repeated tests showed similar results, from all of which the mass spectral peaks can be recognized consistently. These results demonstrate effective in-situ desalting and efficient thermal primer elution.

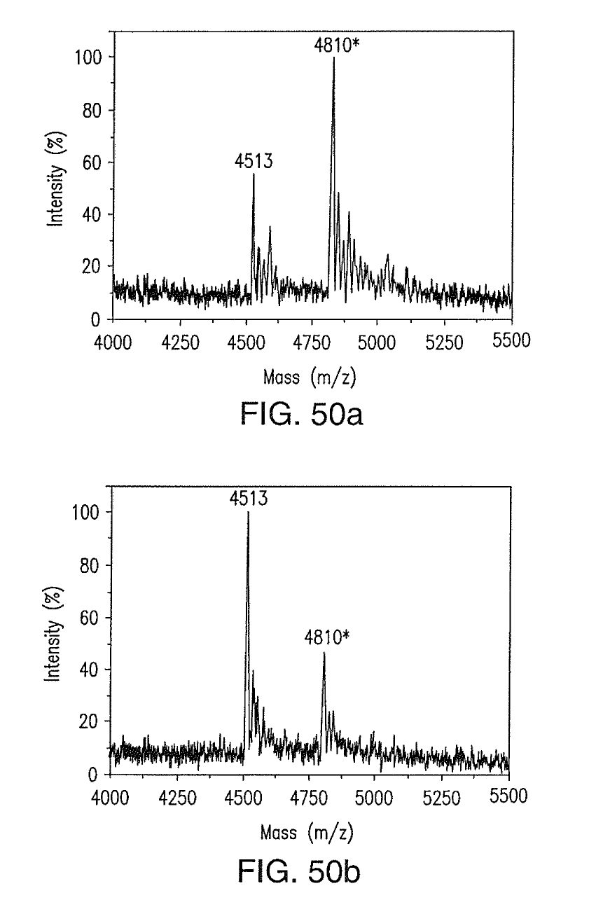

Having tested the individual procedures necessary for SNP detection, the procedures were integrated and the SBE products were analyzed using MALDI-TOF MS. Theoretically, the mass of extended primer can be calculated according to the equation inₚ=mᵣ+mₙ−m_{b}, where mᵣ is the mass of extended primer, mᵣ is the mass of unextended primer, mₙ is the mass of corresponding ddNTP and m_{b} is the mass of bond formation (175 m/z). SNPs on both mutated HBB gene and unmutated HBB gene were detected. As the target nucleotides of the mutated and unmutated template DNA are adenosine and thymidine, a single dideoxyadenosine triphosphate (ddATP, M.W.=472) and dideoxythymidine triphosphate (ddTTP, M.W.=463) were incorporated into each primer, respectively. Thus the mass of the product for mutated and unmutated HBB gene were respectively expected to be 4810 Daltons (4513+472−175), as shown by the distinct peak at 4810 m/z in FIG. 50a, and 4801 Daltons (4513+463−175), as shown by the peak at 4801 m/z in FIG. 50b. The peak located at 4513 m/z in both FIGS. 50a and 50b was induced by unextended primers, which would not compromise the identification of the nucleotide at a SNP site. The repeated genotyping of both mutated and unmutated HBB gene has shown similar mass spectra consistently. Thus, the results indicate successfully integrated SNP detection.

### Example 7

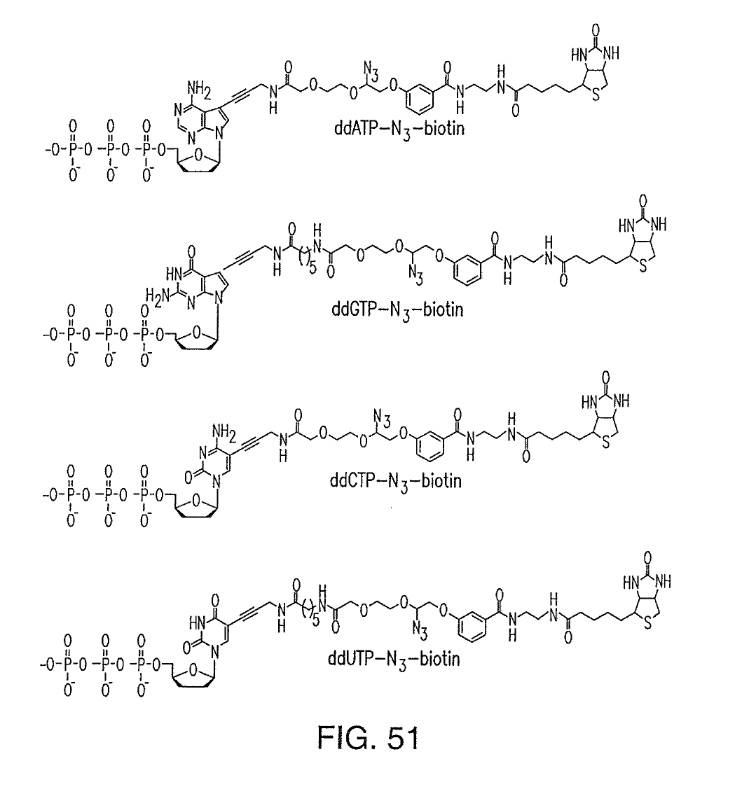

In this example, an alternative approach for detecting polymorphic site of a target DNA, as previously described in connection with FIG. 5, is demonstrated. A number of exemplary molecular structures of cleavable biotinylated ddNTPs, which were used in the Example, are shown in FIG. 51.

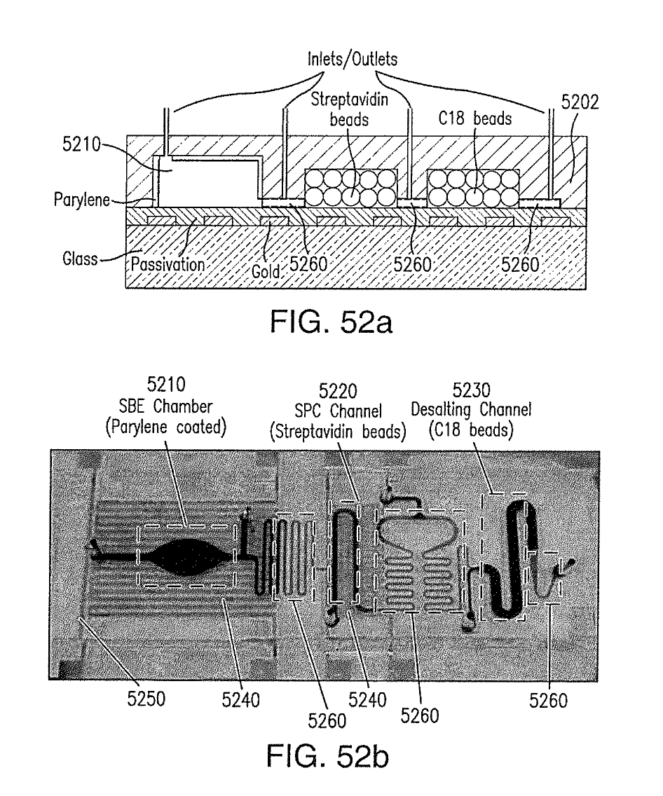

The microdevice used in this Example, as illustrated in FIGS. 52a (schematic) and 52b (image), includes a polydimethyl siloxane (PDMS) sheet 5202 having an SBE chamber 5210, two microchannels (5220 and 5230) for SPC and desalting respectively, and integrated resistive heaters 5240 and temperature sensors 5250 for closed-loop temperature control of the SBE chamber and SPC channel. Between the SBE chamber and the microchannel 5220, and between the microchannels 5220 and 5230, there are weir structures 5260 for retaining the beads loaded in the microchannels.

The device was fabricated using standard microfabrication techniques. Briefly, gold (100 nm) and chrome (5 nm) thin films were thermally evaporated onto the glass substrate, and patterned by photolithography and wet etching. This resulted in resistive temperature sensors and resistive heaters, which were then passivated by deposition of silicon dioxide (1 μm) using plasma enhanced chemical vapor deposition (PECVD). Next, the PDMS sheet was bonded to the temperature control chip irreversibly after treatment with oxygen plasma. Finally, the inner surface of the device was coated with a thin layer of Parylene C via chemical vapor deposition.

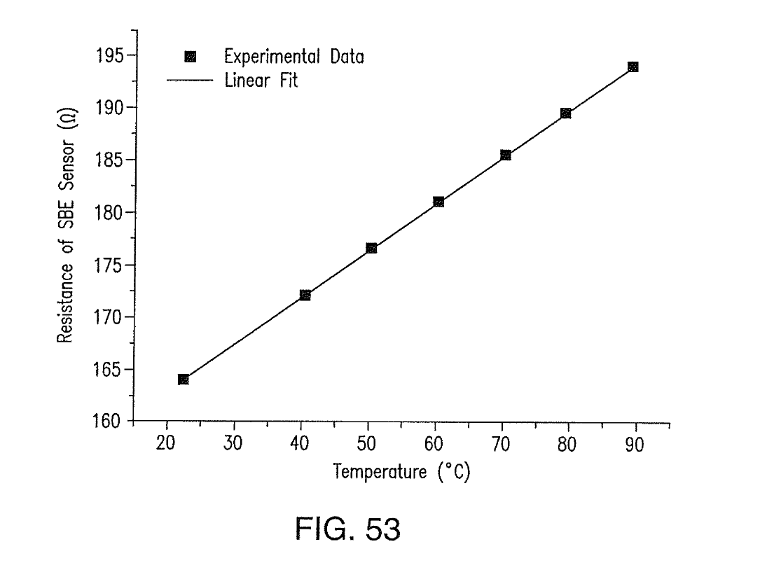

The temperature-resistance relationship of a resistive temperature sensor is calibrated following fabrication to provide accurate temperature control. The resistance of thin film gold resistor is linearly dependent on temperature, as given by R=R₀ (1+α(T−T₀)), where R is the sensor resistance at temperature T, R₀ is the sensor resistance at reference temperature T₀, and α is the sensor's temperature coefficient of resistance (TCR). Measurements of SBE sensor resistances at varying temperatures are shown in FIG. 53, whose highly linear dependence on temperature is in accordance with the above equation. The TCR was calculated to be 2.74×10⁻³ l/° C. The temperature-resistance relationship of the SPC sensor also exhibits linear behavior, with a TCR equal to 2.76×10⁻³ l/° C.

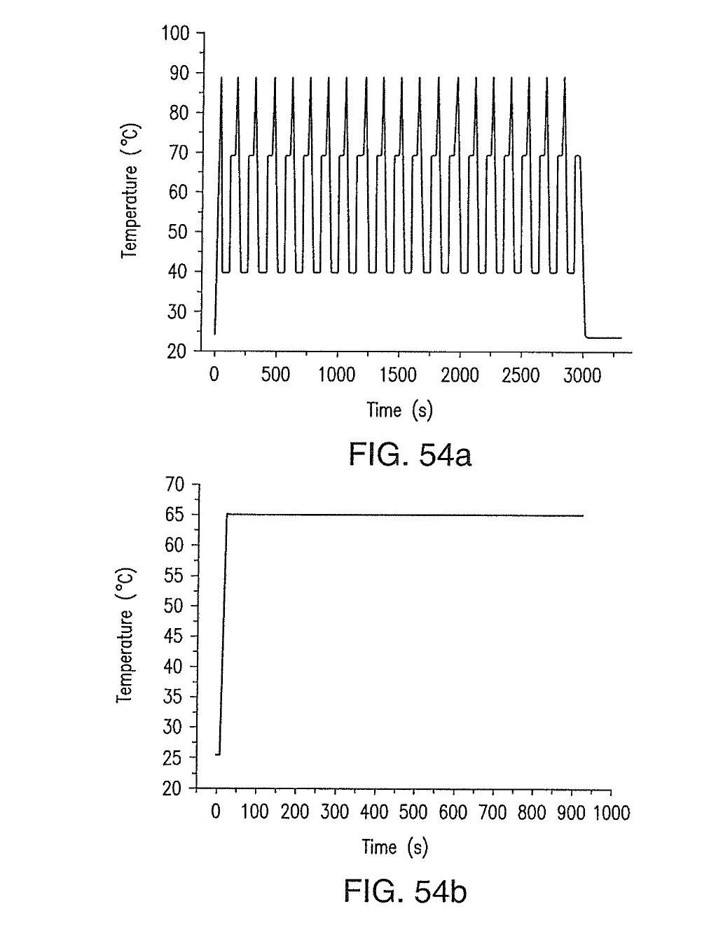

The temperature tracking history was tracked in a test and shown in FIG. 54a, which indicates that the buffer-filled SBE chamber attained the specified temperatures via closed-loop control. The thermal time constants (based on an exponential fit) were 3 s for heating and 11 s for cooling, which represent an improvement over time responses of a conventional PCR thermal cyclers (e.g., 8 s for heating, and 19 s for cooling for Eppendorf Mastercycler® Personal used in related examples below). FIG. 54b shows a temperature history as the buffer-filled SPC channel was heated using closed-loop temperature control. The channel temperature increased from room temperature (25° C.) to 65° C. rapidly in about 13.7 s with an insignificant overshoot 0.25° C.), and then maintained for approximately 15 minutes, which should be sufficient for chemical cleavage-based release of captured SBE products.

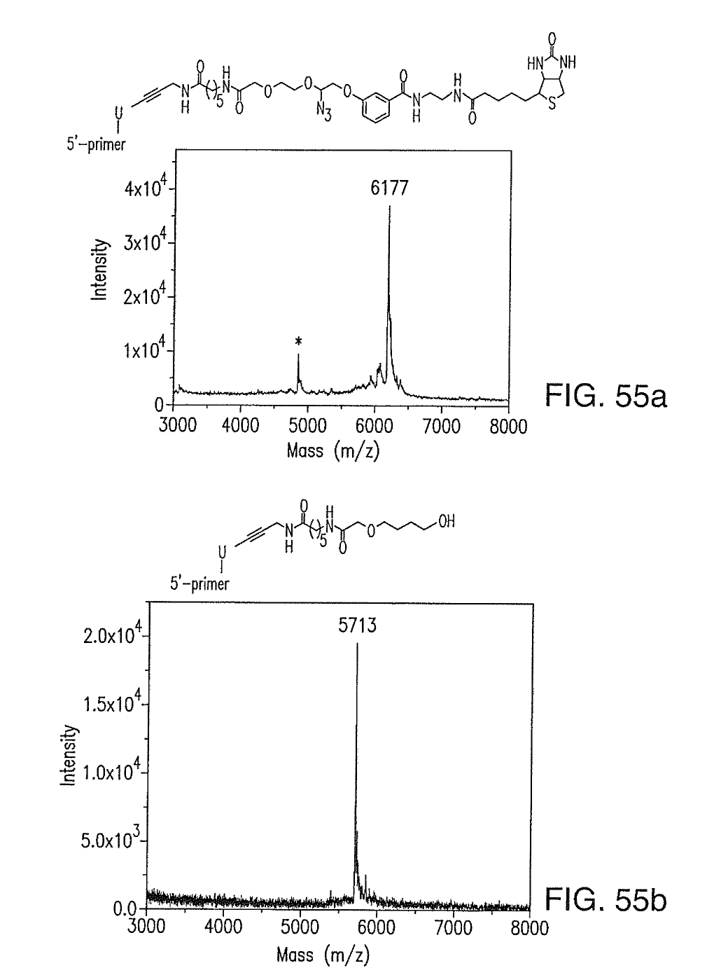

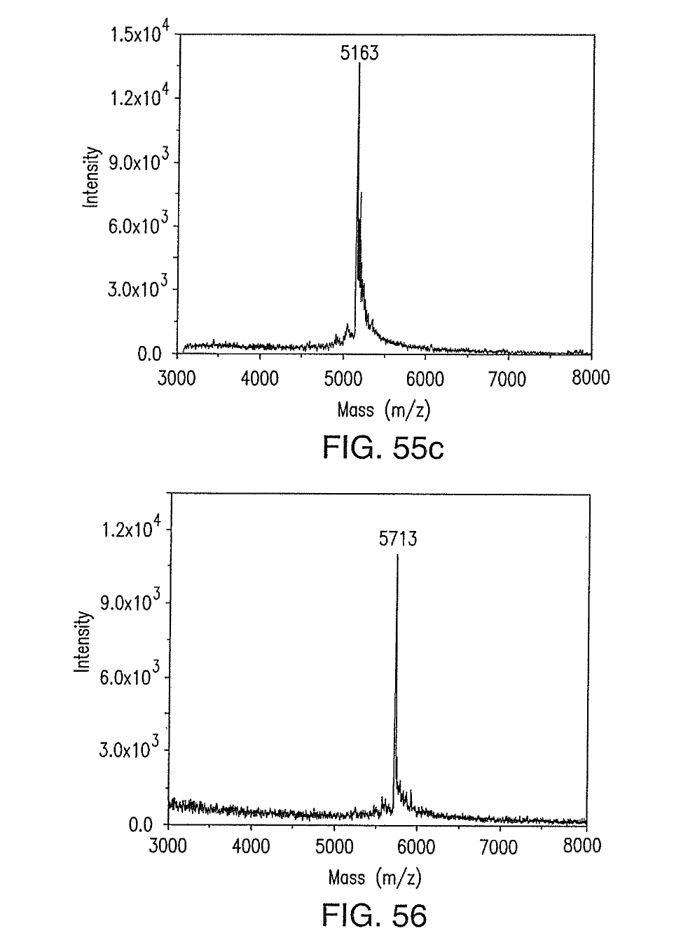

A primer (5′-GATAGGACTCATCACCA-3′, 5163 m/z) (SEQ ID NO:17) targeting exon 8 of the cancer suppressor gene p53 was extended by a single base (ddUTP-N3-biotin) in the SBE chamber. 10 μL of SBE solution was introduced to the SBE chamber and underwent 10 thermal cycles as follows: 90° C. for 10 s, 40° C. for 60 s, 70° C. for 30 s. SBE solution including 20 pmol of synthetic DNA template, 40 pmol of primer, 60 pmol of ddUTP-N3-biotin (M.W.=1189), 1× Thermo Sequenase reaction buffer and 2 units of Thermo Sequenase. Almost 100% of the primer molecules were extended, as shown by a single product peak (6177 m/z) in the mass spectrum (shown in FIG. 55a).

SBE product (10 μL from the commercial thermal cycler, Eppendorf Mastercycler® Personal, using the same SBE solution and parameters) terminated with ddUTP-N3-biotin was successfully captured by streptavidin-coated microbeads packed in the SPC channel and released upon incubation in tris(2-carboxyethyl)phosphine (TCEP, 100 mM, pH=9) at 65° C. for 10 min, as evidenced by a subsequently detected single mass spectral peak at 5713 m/z (FIG. 55b), indicating this approach was capable of capturing biotinylated product and releasing the cleavage product.

To characterize desalting efficiency, as low as 0.5 pmol primer molecules in TCEP were introduced into the C18 bead-packed desalting channel, followed by washing with deionized water and elution with 50% acetonitrile. Effective desalting was observed from a distinct mass spectral peak at 5163 m/z (FIG. 55c), indicating the ability of the microdevice for detecting low concentrations of mutated DNA.

For SNP detection, 10 μL of SBE solution was introduced as before, followed by SBE, SPC, chemical cleavage and desalting in series in the microdevice. The SNP site was detected successfully, as shown in the mass spectrum (FIG. 56), with a single distinct cleavage product peak. This demonstrated that the device was able to perform fully integrated SNP detection as designed.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter.

1. A method for amplifying a target DNA molecule using at least a first microchamber including at least one first primer immobilized on a solid phase in the first microchamber, the first primer suitable for amplifying the target DNA, the method comprising:
(a) introducing a first sample including the target DNA molecule into the first microchamber, whereby the target DNA is hybridized onto the first primer;
(b) producing a complementary DNA of the target DNA in the first microchamber using the target DNA as a template;
(c) separating the target DNA from the complementary DNA;
(d) hybridizing a second primer onto the complementary DNA; and
(e) producing an amplification of the target DNA using the complementary DNA as a template.

2. The method of claim 1, wherein the hybridizing a second primer comprising hybridizing the second primer to a free end of the complementary DNA.

3. The method of claim 1, wherein the second primer comprises a spectroscopically detectable tag.

4. The method of claim 3, wherein the detectable tag comprises a fluorophore.

5. The method of claim 4, further comprising: detecting the target DNA using fluorescent spectroscopy.

6. The method of claim 1, wherein the isolating includes denaturing.

7. The method of claim 1, further comprising repeating (c) through (e) using the second primer to produce a plurality of double-stranded DNA each including a copy of the target DNA and a copy of the complementary DNA.

8. The method of claim 1, wherein the DNA comprises an aptamer.

9. The method of claim 1, wherein the first sample further includes DNA molecules other than the target DNA.

10. The method of claim 1, further comprising obtaining the first sample by:
introducing a second sample including the target DNA and non-target DNA molecules into a second microchamber in fluidic communication with the first microchamber and including an immobilized functional molecule that binds with the target DNA, whereby the target DNA binds with the immobilized functional molecule in the second microchamber;
removing the DNA molecules not bound with the functional molecule; and
isolating the bound target DNA from the functional molecule, thereby obtaining the first sample,
wherein introducing the first sample into the first microchamber includes transporting the first sample from the second microchamber into the first microchamber.

11. The method of claim 10, wherein the target DNA comprises an aptamer.

12. The method of claim 10, wherein the isolating the bound target DNA from the functional molecule includes releasing the bound target DNA at an elevated temperature.

13. The method of claim 10, wherein the isolating the bound target DNA from the functional molecule includes eluting the bound target DNA by a chemical agent solution.

14. The method of claim 10, wherein the transporting includes using electrophoretically transporting the first sample from the second microchamber into the first microchamber via a microchannel connecting the second microchamber into the first microchamber, the microchannel comprising a gel suitable for electrophoresis of the DNA.

15. The method of claim 14, further comprising:
isolating the amplified DNA from the solid phase in the first microchamber; and
electrophoretically transporting the isolated amplified DNA to the second microchamber.

16. The method of claim 1, wherein the target DNA includes at least one polymorphic site, the method further comprising:
isolating the amplified copy of the target DNA from the complementary DNA;
introducing at least one an allele specific primer to anneal adjacent to a site of the complementary DNA corresponding to the at least one polymorphic site;
extending the at least one allele specific primer by one base to obtain an extended primer;
isolating the extended primer from the complementary DNA;
detecting the one base included in the isolated extended primer, thereby determining the identity of the polymorphic site of the target DNA.

17. The method of claim 16, wherein the extending the allele specific primer includes extending the primer in the presence of dideoxynucleotides and a suitable enzyme for the extension.

18. The method of claim 16, wherein the detecting the base included in the isolated extended primer includes detecting the mass of the isolated extended primer using MALDI-TOF mass spectroscopy.

19. The method of claim 16, further comprising: repeating (c) through (e) using the second primer to produce a plurality of double-stranded DNA each including a copy of the target DNA and a copy of the complementary DNA.

20. The method of claim 16, wherein the target DNA includes a plurality of polymorphic sites, and wherein the introducing comprises introducing a plurality of allele specific primers, each to anneal adjacent to one of the plurality of polymorphic sites.

21. The method of any of claim 1, wherein the first microchamber and the second microchamber are formed on a same substrate.

22. A method of isolating and enriching a target DNA using a first microchamber, a second microchamber, and a microchannel connecting the first microchamber and the second microchamber, the microchannel comprising a gel suitable for electrophoresis of the target DNA, comprising:
introducing a sample including the target DNA and non-target DNA molecules into the second microchamber including an immobilized functional molecule that binds with the target DNA, whereby the target DNA binds with the immobilized functional molecule in the first microchamber;
removing the DNA molecules not bound with the functional molecule;
releasing the bound target DNA from the functional molecule;
electrophoretically transporting the released target DNA from the second microchamber into the first microchamber via the microchannel.

23. The method of claim 22, further comprising:
amplifying the target DNA in the first microchamber using a first primer immobilized on a solid phase in the first microchamber.

24. The method of claim 23, further comprising:
isolating the amplified DNA from the solid phase in the first microchamber; and
electrophoretically transporting the isolated amplified DNA to the second microchamber.

25. A method for determining a polymorphic site in a target DNA using a microfluidic device having a first microchamber and a second microchamber in fluidic communication with the first microchamber, comprising:
introducing a sample including the target DNA into the first microchamber;
introducing at least one allele specific primer to anneal immediately adjacent to the polymorphic site of the target DNA;
extending the allele specific primer by one base to obtain an extended primer;
generating a plurality of copies of the extended primer by one or more thermal cycles;
transferring the plurality of copies of the extended primer into the second microchamber which includes a solid phase having surface-attached functional molecules that bind with the extended primer such that the at least one of the plurality of copies of extended primer is captured by the solid phase;
isolating the captured extended primer from the solid phase by chemical cleavage; and
detecting the one base included in the isolated extended primer, thereby determining the identity of the polymorphic site of the target DNA.

26. The method of claim 25, wherein the extending includes extending the primer in the presence of biotinylated dideoxynucleotides.

27. The method of claim 25, wherein the isolating the extended primer from the target DNA includes using a chemical agent solution.

28. The method of claim 25, further comprising, before the detecting, desalting the isolated extended primer.

29. The method of claim 28, wherein the desalting comprises using a microchannel including C18 beads.

30. The method of claim 25, wherein the detecting includes detecting the mass of the isolated extended primer using MALDI-TOF mass spectroscopy.

31. The method of claim 25, wherein the target DNA includes a plurality of polymorphic sites, and wherein the introducing the at least one allele specific primer comprises introducing a plurality of allele specific primers, each to anneal adjacent to one of the plurality of polymorphic sites.

32. A microdevice for amplifying a target DNA, comprising:
a first microchamber formed in a cavity of a multilayered thin film structure,
a solid phase including a plurality of microbeads loaded in the first microchamber, the microbeads including surface immobilized primers suitable for amplifying the target DNA using polymerase chain reaction; and
a temperature regulator thermally coupled to the first microchamber for regulating the temperature of the first microchamber, the temperature regulator comprising a microheater situated in a layer of the multilayered thin film structure proximate the first microchamber.

33. The microdevice of claim 32, further comprising a temperature sensor situated in the same layer of the multilayered thin film structure.

34. The microdevice of claim 32, further comprising a second microchamber in fluidic communication with the first microchamber.

35. The microdevice of claim 34, wherein the second microchamber is connected with the first microchamber via a microchannel, the microchannel comprising a gel suitable for electrophoresis of the target DNA.
